(19) United States
(12) Patent Application Publication
(10) Pub. No.: US 2024/0315184 A1
(43) Pub. Date: Sep. 26, 2024

(54) SNP MOLECULAR MARKER COMBINATION OF BRASSICA NAPUS L. AND APPLICATION METHOD THEREOF

(71) Applicants: Hunan Agricultural University, Changsha (CN); Huazhi Biotech Co., Ltd, Changsha (CN)

(72) Inventors: Zhongsong Liu, Changsha (CN); Le Li, Changsha (CN); Hao Chen, Changsha (CN); Liu Yang, Changsha (CN); Lei Kang, Changsha (CN); Lunwen Qian, Changsha (CN); Gang Xiao, Changsha (CN); Tailong Tan, Changsha (CN); Zhenqian Zhang, Changsha (CN); Yuting Lei, Changsha (CN); Bingchuan Tian, Changsha (CN)

(21) Appl. No.: 18/654,007

(22) Filed: May 3, 2024

Related U.S. Application Data
(63) Continuation of application No. PCT/CN2024/081573, filed on Mar. 14, 2024.

(30) Foreign Application Priority Data
Mar. 21, 2023 (CN) 2023102773520

(51) Int. Cl. A01H 1/04; C12Q 1/6895

(52) U.S. Cl. CPC A01H 1/045; C12Q 1/6895; C12Q 2600/156

(57) ABSTRACT

An SNP molecular marker combination of Brassica napus L. and application method thereof are provided, and the SNP molecular marker combination includes 1407 SNP molecular markers. A set of 1407 high-quality and highly polymorphic SNP markers is screened, which are co-dominant markers, have high specificity, sensitivity and resolution, and have wide application universality. The markers are not affected by environmental conditions and can be detected by using seeds or any type of plant tissue. Detection results are accurate and have good repeatability and stability. Different data results obtained from different testing laboratories can be compared and verified with each other, and the data has universal comparability. A 1.5 K SNP chip is prepared by using the 1407 SNP markers, so as to detect genome-wide SNP of Brassica napus L. samples quickly, with high-throughput and low-cost, which can be effectively used for breeding or assisted breeding of Brassica napus L.

### CROSS-REFERENCE TO RELATED APPLICATION

This application claims priority to Chinese Patent Application No. 202310277352.0, filed Mar. 21, 2023, which is herein incorporated by reference in its entirety.

### TECHNICAL FIELD

The disclosure relates to the technical field of molecular plant breeding, and more particularly to a single nucleotide polymorphism (SNP) molecular marker combination of Brassica napus L. and an application method thereof.

### BACKGROUND

Brassica napus L. (also referred to as rapeseed or oilseed rape) is one of the important oil crops, and is a main source of edible vegetable oil. The Brassica napus L. has a long history of cultivation and utilization, but has a low yield level and poor quality.

In a seed breeding development of the Brassica napus L., existing breeding methods have low efficiency. Therefore, some breeding methods and tools with high efficiency and low cost need to be used to achieve efficient and rapid breeding. The existing technologies supporting Brassica napus L. breeding are mainly sequencing and solid-phase chip detection. The sequencing is a process of detecting a whole genome sequence of a sample, which has high cost. Generally, the cost of the sequencing is more than 500 yuan for a sample, and the sequencing has long cycle of 30 days. Moreover, the amount of data generated in the sequencing is large, which requires professional analysts to process, so it is difficult to popularize in seed breeding production.

### SUMMARY

The disclosure aims to solve at least one of the technical problems in the above related art. Thus, the disclosure proposes a SNP molecular marker combination of Brassica napus L.

The disclosure also proposes a gene chip having the SNP molecular marker combination of Brassica napus L.

The disclosure also proposes application methods of the SNP molecular marker combination of Brassica napus L. and the gene chip.

According to a first aspect, the disclosure proposes a SNP molecular marker combination of Brassica napus L., the SNP molecular marker combination of Brassica napus L. includes 1407 SNP molecular markers, and the 1407 SNP molecular markers are shown in BN0001-1407. Physical positions of the 1407 SNP molecular markers are determined by sequence alignment based on a reference genome Brana_Dar_ViG (can be found in Chalhoub, B. et al. Plant genetics. Early allopolyploid evolution in the post-Neolithic Brassica napus oilseed genome. Science 345, 950-953 (2014)) of Brassica napus L., and specific locus information of the 1407 SNP molecular markers is as follows:

| Maeker | SNP | | Physical |
|---|---|---|---|
| name | locus | Chromosome | position |
| BN0001 | [A/G] | A01 | 129037 |
| BN0002 | [G/T] | A01 | 428925 |
| BN0003 | [A/T] | A01 | 613260 |
| BN0004 | [G/C] | A01 | 766699 |
| BN0005 | [G/A] | A01 | 1025737 |
| BN0006 | [C/T] | A01 | 1437661 |
| BN0007 | [A/G] | A01 | 1836189 |
| BN0008 | [G/C] | A01 | 2101529 |
| BN0009 | [T/C] | A01 | 2621155 |
| BN0010 | [C/T] | A01 | 3189305 |
| BN0011 | [A/G] | A01 | 3484531 |
| BN0012 | [C/A] | A01 | 3915906 |
| BN0013 | [A/G] | A01 | 4798105 |
| BN0014 | [A/G] | A01 | 4995490 |
| BN0015 | [G/C] | A01 | 5656374 |
| BN0016 | [T/C] | A01 | 6256801 |
| BN0017 | [G/T] | A01 | 6554695 |
| BN0018 | [C/G] | A01 | 6975952 |
| BN0019 | [A/G] | A01 | 7256244 |
| BN0020 | [G/A] | A01 | 8082266 |
| BN0021 | [T/A] | A01 | 8422189 |
| BN0022 | [T/A] | A01 | 8580582 |
| BN0023 | [C/G] | A01 | 9496906 |
| BN0024 | [G/A] | A01 | 9964538 |
| BN0025 | [T/G] | A01 | 10278462 |
| BN0026 | [T/C] | A01 | 10691388 |
| BN0027 | [C/T] | A01 | 10914494 |
| BN0028 | [C/A] | A01 | 11735978 |
| BN0029 | [C/T] | A01 | 12075653 |
| BN0030 | [C/T] | A01 | 13215910 |
| BN0031 | [T/A] | A01 | 13369626 |
| BN0032 | [T/A] | A01 | 13792030 |
| BN0033 | [C/G] | A01 | 14076059 |
| BN0034 | [T/A] | A01 | 14242025 |
| BN0035 | [A/G] | A01 | 14531087 |
| BN0036 | [C/T] | A01 | 14591462 |
| BN0037 | [G/T] | A01 | 14820924 |
| BN0038 | [G/C] | A01 | 15092908 |
| BN0039 | [T/C] | A01 | 15384809 |
| BN0040 | [G/T] | A01 | 15676588 |
| BN0041 | [T/A] | A01 | 15908466 |
| BN0042 | [A/G] | A01 | 16165301 |
| BN0043 | [C/T] | A01 | 17080618 |
| BN0044 | [G/A] | A01 | 17831711 |
| BN0045 | [A/C] | A01 | 17938462 |
| BN0046 | [T/G] | A01 | 18314187 |
| BN0047 | [C/T] | A01 | 18477965 |
| BN0048 | [T/C] | A01 | 19157860 |
| BN0049 | [A/G] | A01 | 19811843 |
| BN0050 | [C/T] | A01 | 20761418 |
| BN0051 | [T/C] | A01 | 21872829 |
| BN0052 | [T/C] | A01 | 22498537 |
| BN0053 | [G/A] | A01 | 23055560 |
| BN0054 | [T/C] | A01 | 23481834 |
| BN0055 | [G/C] | A01 | 23789453 |
| BN0056 | [T/A] | A01 | 24071025 |
| BN0057 | [G/A] | A01 | 25164508 |
| BN0058 | [G/A] | A01 | 25312032 |
| BN0059 | [T/A] | A01 | 26099200 |
| BN0060 | [T/C] | A01 | 26704687 |
| BN0061 | [C/T] | A01 | 27108445 |
| BN0062 | [T/C] | A01 | 27500324 |
| BN0063 | [T/G] | A01 | 27754935 |
| BN0064 | [G/T] | A01 | 28259675 |
| BN0065 | [G/T] | A01 | 28940588 |
| BN0066 | [C/T] | A01 | 29186931 |
| BN0067 | [T/G] | A01 | 29586142 |
| BN0068 | [T/A] | A01 | 29799486 |
| BN0069 | [T/A] | A01 | 30473575 |
| BN0070 | [A/C] | A01 | 30822101 |
| BN0071 | [G/C] | A01 | 30991803 |
| BN0072 | [C/T] | A01 | 31286361 |
| BN0073 | [G/T] | A01 | 32753867 |
| BN0074 | [C/G] | A01 | 32920903 |
| BN0075 | [T/A] | A02 | 1477438 |
| BN0076 | [A/G] | A02 | 1706353 |
| BN0077 | [T/C] | A02 | 2202267 |
| BN0078 | [A/T] | A02 | 2647003 |
| BN0079 | [G/T] | A02 | 2880803 |
| BN0080 | [G/A] | A02 | 3337050 |
| BN0081 | [C/T] | A02 | 3537064 |
| BN0082 | [T/C] | A02 | 3742268 |
| BN0083 | [A/C] | A02 | 4037886 |
| BN0084 | [G/C] | A02 | 5199998 |
| BN0085 | [T/C] | A02 | 5422396 |
| BN0086 | [T/C] | A02 | 5771631 |
| BN0087 | [G/A] | A02 | 5994344 |
| BN0088 | [C/T] | A02 | 6247196 |
| BN0089 | [G/C] | A02 | 6536986 |
| BN0090 | [A/C] | A02 | 6818209 |
| BN0091 | [G/C] | A02 | 7142397 |
| BN0092 | [A/G] | A02 | 7416706 |
| BN0093 | [T/A] | A02 | 7939578 |
| BN0094 | [T/C] | A02 | 8157741 |
| BN0095 | [T/G] | A02 | 8755245 |
| BN0096 | [C/T] | A02 | 9028967 |
| BN0097 | [T/C] | A02 | 9629117 |
| BN0098 | [G/A] | A02 | 10535585 |
| BN0099 | [G/A] | A02 | 10911720 |
| BN0100 | [A/C] | A02 | 11254116 |
| BN0101 | [A/G] | A02 | 11526796 |
| BN0102 | [T/C] | A02 | 11950079 |
| BN0103 | [T/C] | A02 | 12672987 |
| BN0104 | [T/C] | A02 | 13136845 |
| BN0105 | [C/T] | A02 | 13651745 |
| BN0106 | [G/T] | A02 | 14111450 |
| BN0107 | [C/T] | A02 | 14715499 |
| BN0108 | [A/T] | A02 | 16173502 |
| BN0109 | [A/G] | A02 | 16525868 |
| BN0110 | [T/C] | A02 | 17111749 |
| BN0111 | [G/T] | A02 | 17460550 |
| BN0112 | [A/G] | A02 | 17715705 |
| BN0113 | [T/A] | A02 | 18015759 |
| BN0114 | [T/A] | A02 | 18243304 |
| BN0115 | [A/C] | A02 | 18489557 |
| BN0116 | [G/A] | A02 | 18811701 |
| BN0117 | [T/C] | A02 | 19028233 |
| BN0118 | [A/C] | A02 | 19496673 |
| BN0119 | [G/T] | A02 | 19729156 |
| BN0120 | [A/G] | A02 | 20966584 |
| BN0121 | [T/G] | A02 | 21300405 |
| BN0122 | [G/A] | A02 | 21502165 |
| BN0123 | [C/T] | A02 | 21776475 |
| BN0124 | [G/A] | A02 | 21992906 |
| BN0125 | [G/T] | A02 | 22188583 |
| BN0126 | [A/G] | A02 | 22381160 |
| BN0127 | [G/A] | A02 | 22940568 |
| BN0128 | [C/T] | A02 | 23457378 |
| BN0129 | [A/G] | A02 | 24005725 |
| BN0130 | [T/C] | A02 | 26214476 |
| BN0131 | [T/C] | A02 | 27131996 |
| BN0132 | [G/A] | A02 | 27732157 |
| BN0133 | [C/A] | A02 | 28695870 |
| BN0134 | [C/A] | A02 | 29661805 |
| BN0135 | [C/T] | A02 | 29930522 |
| BN0136 | [C/T] | A02 | 31050927 |
| BN0137 | [C/A] | A02 | 31409538 |
| BN0138 | [G/C] | A02 | 32880693 |
| BN0139 | [A/G] | A03 | 406149 |
| BN0140 | [A/T] | A03 | 614038 |
| BN0141 | [C/T] | A03 | 1770044 |
| BN0142 | [A/C] | A03 | 2321281 |
| BN0143 | [C/T] | A03 | 2655754 |
| BN0144 | [G/C] | A03 | 2866250 |
| BN0145 | [A/G] | A03 | 3186370 |
| BN0146 | [A/T] | A03 | 3778663 |
| BN0147 | [T/C] | A03 | 4062184 |
| BN0148 | [C/A] | A03 | 4299121 |
| BN0149 | [A/T] | A03 | 4528203 |
| BN0150 | [A/C] | A03 | 5174561 |
| BN0151 | [T/G] | A03 | 5484353 |
| BN0152 | [C/T] | A03 | 5752271 |
| BN0153 | [T/C] | A03 | 6715834 |
| BN0154 | [C/T] | A03 | 7407617 |
| BN0155 | [G/A] | A03 | 8844122 |
| BN0156 | [A/G] | A03 | 9326938 |
| BN0157 | [G/A] | A03 | 9801889 |
| BN0158 | [A/G] | A03 | 10152463 |
| BN0159 | [T/C] | A03 | 11478790 |
| BN0160 | [G/A] | A03 | 11727707 |
| BN0161 | [T/C] | A03 | 12109548 |
| BN0162 | [G/T] | A03 | 13024780 |
| BN0163 | [G/A] | A03 | 13567802 |
| BN0164 | [A/G] | A03 | 13959325 |
| BN0165 | [T/C] | A03 | 14275400 |
| BN0166 | [G/A] | A03 | 15788344 |
| BN0167 | [A/G] | A03 | 16465433 |
| BN0168 | [T/C] | A03 | 16728477 |
| BN0169 | [G/C] | A03 | 17059091 |
| BN0170 | [A/C] | A03 | 18279238 |
| BN0171 | [A/G] | A03 | 19095438 |
| BN0172 | [C/G] | A03 | 19716647 |
| BN0173 | [A/T] | A03 | 19959329 |
| BN0174 | [T/C] | A03 | 20562498 |
| BN0175 | [T/C] | A03 | 20997431 |
| BN0176 | [C/G] | A03 | 21668732 |
| BN0177 | [C/T] | A03 | 22160666 |
| BN0178 | [C/T] | A03 | 23469478 |
| BN0179 | [A/G] | A03 | 23854578 |
| BN0180 | [A/T] | A03 | 24268573 |
| BN0181 | [T/C] | A03 | 24618632 |
| BN0182 | [A/G] | A03 | 25024759 |
| BN0183 | [A/G] | A03 | 25502419 |
| BN0184 | [T/G] | A03 | 26062918 |
| BN0185 | [A/G] | A03 | 26781654 |
| BN0186 | [T/G] | A03 | 27289278 |
| BN0187 | [G/C] | A03 | 27644223 |
| BN0188 | [T/C] | A03 | 27920290 |
| BN0189 | [G/A] | A03 | 28624068 |
| BN0190 | [C/T] | A03 | 29181558 |
| BN0191 | [G/A] | A03 | 29521804 |
| BN0192 | [G/A] | A03 | 29732498 |
| BN0193 | [A/G] | A03 | 30075229 |
| BN0194 | [G/T] | A03 | 30399550 |
| BN0195 | [A/G] | A03 | 30882866 |
| BN0196 | [T/C] | A03 | 31559217 |
| BN0197 | [C/G] | A03 | 31977200 |
| BN0198 | [T/G] | A03 | 32271145 |
| BN0199 | [G/A] | A03 | 33680884 |
| BN0200 | [T/C] | A03 | 35229675 |
| BN0201 | [C/T] | A03 | 36037542 |
| BN0202 | [G/C] | A03 | 36544390 |
| BN0203 | [A/T] | A03 | 37502943 |
| BN0204 | [G/C] | A04 | 605905 |
| BN0205 | [G/T] | A04 | 1516508 |
| BN0206 | [A/T] | A04 | 2817214 |
| BN0207 | [C/T] | A04 | 3084717 |
| BN0208 | [C/T] | A04 | 3665795 |
| BN0209 | [A/G] | A04 | 4365699 |
| BN0210 | [T/C] | A04 | 5021135 |
| BN0211 | [C/A] | A04 | 5210952 |
| BN0212 | [A/G] | A04 | 6339746 |
| BN0213 | [T/G] | A04 | 6975258 |
| BN0214 | [T/C] | A04 | 7783900 |
| BN0215 | [A/G] | A04 | 9325873 |
| BN0216 | [C/A] | A04 | 10357480 |
| BN0217 | [G/T] | A04 | 11134891 |
| BN0218 | [C/T] | A04 | 11793239 |
| BN0219 | [G/A] | A04 | 12433336 |
| BN0220 | [G/A] | A04 | 12752347 |
| BN0221 | [A/G] | A04 | 13213440 |
| BN0222 | [T/C] | A04 | 13585869 |
| BN0223 | [A/G] | A04 | 14061987 |
| BN0224 | [C/T] | A04 | 14699418 |
| BN0225 | [C/T] | A04 | 15810172 |
| BN0226 | [G/A] | A04 | 16551694 |
| BN0227 | [G/T] | A04 | 17086259 |
| BN0228 | [A/G] | A04 | 17366895 |
| BN0229 | [C/G] | A04 | 18673865 |
| BN0230 | [G/A] | A04 | 19109792 |
| BN0231 | [A/G] | A04 | 19438862 |
| BN0232 | [C/T] | A04 | 19516841 |
| BN0233 | [A/G] | A04 | 19894101 |
| BN0234 | [G/A] | A04 | 20296015 |
| BN0235 | [C/A] | A04 | 20678658 |
| BN0236 | [C/T] | A04 | 21001498 |
| BN0237 | [C/T] | A04 | 21427386 |
| BN0238 | [G/A] | A04 | 22221101 |
| BN0239 | [C/T] | A04 | 22397348 |
| BN0240 | [A/G] | A05 | 10899 |
| BN0241 | [A/G] | A05 | 390148 |
| BN0242 | [G/T] | A05 | 582316 |
| BN0243 | [A/G] | A05 | 1086270 |
| BN0244 | [A/G] | A05 | 1811198 |
| BN0245 | [C/T] | A05 | 1924532 |
| BN0246 | [G/A] | A05 | 2661901 |
| BN0247 | [A/T] | A05 | 2841541 |
| BN0248 | [C/T] | A05 | 4323400 |
| BN0249 | [T/C] | A05 | 5009580 |
| BN0250 | [C/G] | A05 | 5499857 |
| BN0251 | [A/G] | A05 | 6977523 |
| BN0252 | [T/C] | A05 | 7372610 |
| BN0253 | [T/C] | A05 | 8749499 |
| BN0254 | [C/A] | A05 | 8959263 |
| BN0255 | [G/A] | A05 | 10258530 |
| BN0256 | [T/G] | A05 | 11564316 |
| BN0257 | [C/G] | A05 | 11883288 |
| BN0258 | [A/G] | A05 | 12452356 |
| BN0259 | [A/C] | A05 | 13061358 |
| BN0260 | [G/C] | A05 | 13258563 |
| BN0261 | [A/T] | A05 | 13491544 |
| BN0262 | [G/A] | A05 | 13553669 |
| BN0263 | [G/T] | A05 | 15101609 |
| BN0264 | [A/G] | A05 | 16208236 |
| BN0265 | [C/T] | A05 | 17277143 |
| BN0266 | [C/A] | A05 | 17491278 |
| BN0267 | [G/C] | A05 | 19577916 |
| BN0268 | [G/A] | A05 | 19958998 |
| BN0269 | [C/T] | A05 | 20998579 |
| BN0270 | [C/T] | A05 | 25415343 |
| BN0271 | [A/C] | A05 | 26386555 |
| BN0272 | [G/A] | A05 | 26590152 |
| BN0273 | [C/T] | A05 | 27379626 |
| BN0274 | [A/T] | A05 | 27731387 |
| BN0275 | [C/T] | A05 | 28069804 |
| BN0276 | [G/A] | A05 | 28481737 |
| BN0277 | [C/T] | A05 | 29107210 |
| BN0278 | [C/T] | A05 | 30788919 |
| BN0279 | [C/T] | A05 | 31816944 |
| BN0280 | [A/G] | A05 | 32603435 |
| BN0281 | [G/A] | A05 | 33110405 |
| BN0282 | [C/G] | A05 | 33357533 |
| BN0283 | [A/G] | A05 | 34099497 |
| BN0284 | [A/C] | A05 | 34341577 |
| BN0285 | [T/C] | A05 | 34663148 |
| BN0286 | [A/C] | A05 | 35608329 |
| BN0287 | [C/T] | A05 | 35829658 |
| BN0288 | [G/A] | A05 | 36312828 |
| BN0289 | [C/A] | A05 | 36600227 |
| BN0290 | [A/T] | A05 | 37611553 |
| BN0291 | [T/G] | A05 | 38108829 |
| BN0292 | [A/G] | A05 | 38865463 |
| BN0293 | [C/A] | A05 | 39209611 |
| BN0294 | [A/T] | A05 | 40839055 |
| BN0295 | [G/A] | A05 | 41820634 |
| BN0296 | [C/A] | A06 | 129694 |
| BN0297 | [A/G] | A06 | 1567300 |
| BN0298 | [A/G] | A06 | 3130615 |
| BN0299 | [G/T] | A06 | 3605047 |
| BN0300 | [T/A] | A06 | 3859392 |
| BN0301 | [A/G] | A06 | 5116532 |
| BN0302 | [G/A] | A06 | 5366079 |
| BN0303 | [C/T] | A06 | 5642020 |
| BN0304 | [C/A] | A06 | 5926997 |
| BN0305 | [A/G] | A06 | 6472206 |
| BN0306 | [G/A] | A06 | 6710627 |
| BN0307 | [G/A] | A06 | 7680229 |
| BN0308 | [T/A] | A06 | 7877587 |
| BN0309 | [C/T] | A06 | 8506048 |
| BN0310 | [T/G] | A06 | 8893548 |
| BN0311 | [A/G] | A06 | 9127109 |
| BN0312 | [T/C] | A06 | 9759098 |
| BN0313 | [A/G] | A06 | 10041482 |
| BN0314 | [A/C] | A06 | 10381729 |
| BN0315 | [G/A] | A06 | 10659873 |
| BN0316 | [C/A] | A06 | 11032570 |
| BN0317 | [T/G] | A06 | 12034384 |
| BN0318 | [A/G] | A06 | 12272687 |
| BN0319 | [A/C] | A06 | 12613541 |
| BN0320 | [C/A] | A06 | 13203632 |
| BN0321 | [C/A] | A06 | 13734879 |
| BN0322 | [T/C] | A06 | 14356865 |
| BN0323 | [G/A] | A06 | 14874993 |
| BN0324 | [A/G] | A06 | 15045436 |
| BN0325 | [G/A] | A06 | 15305898 |
| BN0326 | [G/T] | A06 | 17550566 |
| BN0327 | [A/G] | A06 | 22600936 |
| BN0328 | [G/T] | A06 | 22883532 |
| BN0329 | [T/C] | A06 | 23682059 |
| BN0330 | [A/G] | A06 | 23737247 |
| BN0331 | [C/G] | A06 | 24238616 |
| BN0332 | [G/T] | A06 | 25517201 |
| BN0333 | [C/A] | A06 | 26347762 |
| BN0334 | [G/C] | A06 | 26648095 |
| BN0335 | [G/A] | A06 | 26802195 |
| BN0336 | [T/C] | A06 | 28207078 |
| BN0337 | [C/G] | A06 | 31270202 |
| BN0338 | [G/A] | A06 | 31570643 |
| BN0339 | [T/G] | A06 | 31833475 |
| BN0340 | [G/A] | A06 | 32052205 |
| BN0341 | [G/A] | A06 | 32843815 |
| BN0342 | [C/T] | A06 | 33508966 |
| BN0343 | [C/T] | A06 | 34300539 |
| BN0344 | [G/A] | A06 | 34469363 |
| BN0345 | [G/T] | A06 | 34832781 |
| BN0346 | [G/T] | A06 | 35268689 |
| BN0347 | [A/C] | A06 | 35895088 |
| BN0348 | [G/A] | A06 | 36240436 |
| BN0349 | [A/G] | A06 | 36750388 |
| BN0350 | [A/G] | A06 | 36978489 |
| BN0351 | [G/A] | A06 | 37325744 |
| BN0352 | [T/C] | A06 | 37579242 |
| BN0353 | [G/A] | A06 | 38576970 |
| BN0354 | [T/C] | A06 | 39088036 |
| BN0355 | [A/G] | A06 | 39303110 |
| BN0356 | [T/C] | A06 | 39645025 |
| BN0357 | [C/G] | A06 | 40659271 |
| BN0358 | [G/T] | A06 | 41278454 |
| BN0359 | [G/A] | A06 | 41495428 |
| BN0360 | [T/G] | A06 | 41948893 |
| BN0361 | [G/A] | A06 | 42246097 |
| BN0362 | [T/C] | A06 | 42596937 |
| BN0363 | [T/C] | A06 | 44269738 |
| BN0364 | [C/A] | A07 | 668085 |
| BN0365 | [A/G] | A07 | 2171771 |
| BN0366 | [G/C] | A07 | 2862401 |
| BN0367 | [G/C] | A07 | 2965262 |
| BN0368 | [T/C] | A07 | 3217388 |
| BN0369 | [A/G] | A07 | 3565365 |
| BN0370 | [A/G] | A07 | 3738488 |
| BN0371 | [A/T] | A07 | 4040624 |
| BN0372 | [G/A] | A07 | 4514906 |
| BN0373 | [C/G] | A07 | 5125677 |
| BN0374 | [A/T] | A07 | 5550115 |
| BN0375 | [T/G] | A07 | 6234141 |
| BN0376 | [A/C] | A07 | 7290359 |
| BN0377 | [C/T] | A07 | 7692692 |
| BN0378 | [T/C] | A07 | 7937632 |
| BN0379 | [T/A] | A07 | 8276541 |
| BN0380 | [G/A] | A07 | 9044074 |
| BN0381 | [C/T] | A07 | 9815556 |
| BN0382 | [C/G] | A07 | 10425619 |
| BN0383 | [T/C] | A07 | 11527525 |
| BN0384 | [G/T] | A07 | 11978225 |
| BN0385 | [A/T] | A07 | 12115653 |
| BN0386 | [T/A] | A07 | 12854015 |
| BN0387 | [C/T] | A07 | 13237954 |
| BN0388 | [G/A] | A07 | 13893669 |
| BN0389 | [C/T] | A07 | 14324741 |
| BN0390 | [C/T] | A07 | 14784694 |
| BN0391 | [G/A] | A07 | 15226791 |
| BN0392 | [A/T] | A07 | 15898732 |
| BN0393 | [C/A] | A07 | 17352868 |
| BN0394 | [A/G] | A07 | 18072617 |
| BN0395 | [C/A] | A07 | 19546201 |
| BN0396 | [G/A] | A07 | 20348366 |
| BN0397 | [T/C] | A07 | 20718232 |
| BN0398 | [G/A] | A07 | 21442948 |
| BN0399 | [T/G] | A07 | 21729267 |
| BN0400 | [T/C] | A07 | 22066928 |
| BN0401 | [T/C] | A07 | 22482144 |
| BN0402 | [C/A] | A07 | 23044434 |
| BN0403 | [C/T] | A07 | 23347169 |
| BN0404 | [C/T] | A07 | 23969864 |
| BN0405 | [G/A] | A07 | 24340883 |
| BN0406 | [C/T] | A07 | 24707123 |
| BN0407 | [G/T] | A07 | 25187263 |
| BN0408 | [C/G] | A07 | 25478920 |
| BN0409 | [G/A] | A07 | 25894147 |
| BN0410 | [C/A] | A07 | 26262783 |
| BN0411 | [A/G] | A07 | 26589215 |
| BN0412 | [G/A] | A07 | 26845711 |
| BN0413 | [T/C] | A07 | 27000458 |
| BN0414 | [A/T] | A07 | 27698572 |
| BN0415 | [A/G] | A07 | 28244204 |
| BN0416 | [A/G] | A07 | 28937837 |
| BN0417 | [A/T] | A07 | 29275848 |
| BN0418 | [C/T] | A08 | 631906 |
| BN0419 | [A/G] | A08 | 779607 |
| BN0420 | [T/A] | A08 | 1123921 |
| BN0421 | [A/G] | A08 | 1856707 |
| BN0422 | [C/T] | A08 | 2233185 |
| BN0423 | [G/A] | A08 | 2485603 |
| BN0424 | [G/A] | A08 | 2666843 |
| BN0425 | [T/A] | A08 | 3414886 |
| BN0426 | [A/G] | A08 | 4098983 |
| BN0427 | [C/T] | A08 | 4312251 |
| BN0428 | [C/T] | A08 | 5696369 |
| BN0429 | [G/A] | A08 | 6440305 |
| BN0430 | [C/T] | A08 | 7424918 |
| BN0431 | [G/A] | A08 | 7569419 |
| BN0432 | [G/A] | A08 | 8046074 |
| BN0433 | [C/A] | A08 | 9018021 |
| BN0434 | [G/C] | A08 | 9907323 |
| BN0435 | [A/G] | A08 | 10220612 |
| BN0436 | [C/A] | A08 | 10582809 |
| BN0437 | [T/C] | A08 | 11589004 |
| BN0438 | [T/G] | A08 | 11899471 |
| BN0439 | [A/G] | A08 | 12279983 |
| BN0440 | [C/G] | A08 | 13205651 |
| BN0441 | [C/G] | A08 | 13430201 |
| BN0442 | [T/A] | A08 | 13718301 |
| BN0443 | [G/A] | A08 | 13949898 |
| BN0444 | [G/A] | A08 | 14181039 |
| BN0445 | [T/C] | A08 | 14496188 |
| BN0446 | [T/G] | A08 | 14723327 |
| BN0447 | [T/C] | A08 | 15030289 |
| BN0448 | [G/A] | A08 | 15778329 |
| BN0449 | [G/C] | A08 | 16072164 |
| BN0450 | [T/C] | A08 | 16616518 |
| BN0451 | [G/A] | A08 | 17376251 |
| BN0452 | [A/T] | A08 | 17698239 |
| BN0453 | [T/A] | A08 | 18007170 |
| BN0454 | [G/A] | A08 | 18963249 |
| BN0455 | [G/A] | A08 | 19251338 |
| BN0456 | [G/C] | A08 | 19670443 |
| BN0457 | [G/A] | A08 | 20006619 |
| BN0458 | [A/G] | A08 | 20166166 |
| BN0459 | [C/A] | A08 | 20962898 |
| BN0460 | [T/C] | A08 | 21241059 |
| BN0461 | [G/A] | A08 | 21955248 |
| BN0462 | [C/T] | A08 | 22433691 |
| BN0463 | [G/C] | A08 | 23418525 |
| BN0464 | [C/T] | A08 | 23772405 |
| BN0465 | [C/T] | A08 | 24056759 |
| BN0466 | [A/G] | A08 | 24439272 |
| BN0467 | [C/G] | A08 | 24855357 |
| BN0468 | [C/T] | A08 | 25528663 |
| BN0469 | [A/T] | A09 | 683941 |
| BN0470 | [A/G] | A09 | 1444300 |
| BN0471 | [T/C] | A09 | 2168403 |
| BN0472 | [T/C] | A09 | 2575381 |
| BN0473 | [G/T] | A09 | 2991392 |
| BN0474 | [G/A] | A09 | 3356668 |
| BN0475 | [T/A] | A09 | 4172077 |
| BN0476 | [C/T] | A09 | 4945897 |
| BN0477 | [G/A] | A09 | 6184467 |
| BN0478 | [T/C] | A09 | 6479356 |
| BN0479 | [T/C] | A09 | 6860948 |
| BN0480 | [T/A] | A09 | 7148472 |
| BN0481 | [C/T] | A09 | 7345802 |
| BN0482 | [G/T] | A09 | 7901623 |
| BN0483 | [C/T] | A09 | 8256377 |
| BN0484 | [T/G] | A09 | 8902282 |
| BN0485 | [C/T] | A09 | 9351158 |
| BN0486 | [G/A] | A09 | 9891239 |
| BN0487 | [A/G] | A09 | 11033545 |
| BN0488 | [A/C] | A09 | 11299500 |
| BN0489 | [A/G] | A09 | 11558775 |
| BN0490 | [A/G] | A09 | 12223445 |
| BN0491 | [T/C] | A09 | 12407616 |
| BN0492 | [T/C] | A09 | 12708854 |
| BN0493 | [C/A] | A09 | 13404932 |
| BN0494 | [T/A] | A09 | 13740653 |
| BN0495 | [A/G] | A09 | 14589940 |
| BN0496 | [C/A] | A09 | 16414756 |
| BN0497 | [T/C] | A09 | 17591969 |
| BN0498 | [G/A] | A09 | 18102674 |
| BN0499 | [A/T] | A09 | 18503422 |
| BN0500 | [C/T] | A09 | 19728271 |
| BN0501 | [C/T] | A09 | 20030529 |
| BN0502 | [G/C] | A09 | 20374382 |
| BN0503 | [G/A] | A09 | 20620568 |
| BN0504 | [A/G] | A09 | 20910738 |
| BN0505 | [C/T] | A09 | 21142497 |
| BN0506 | [G/A] | A09 | 22780701 |
| BN0507 | [G/A] | A09 | 25604964 |
| BN0508 | [C/T] | A09 | 27068826 |
| BN0509 | [A/T] | A09 | 28187150 |
| BN0510 | [T/C] | A09 | 29227077 |
| BN0511 | [A/T] | A09 | 30357361 |
| BN0512 | [T/A] | A09 | 30995903 |
| BN0513 | [A/C] | A09 | 31291528 |
| BN0514 | [C/T] | A09 | 31496175 |
| BN0515 | [A/G] | A09 | 31852632 |
| BN0516 | [A/G] | A09 | 32180423 |
| BN0517 | [C/G] | A09 | 32521467 |
| BN0518 | [T/G] | A09 | 32701933 |
| BN0519 | [A/C] | A09 | 32973381 |
| BN0520 | [C/T] | A09 | 33322885 |
| BN0521 | [G/A] | A09 | 34222096 |
| BN0522 | [C/A] | A09 | 34563605 |
| BN0523 | [T/C] | A09 | 35114800 |
| BN0524 | [C/T] | A09 | 35473201 |
| BN0525 | [C/A] | A09 | 36422923 |
| BN0526 | [C/T] | A09 | 37285873 |
| BN0527 | [C/A] | A09 | 38228054 |
| BN0528 | [G/A] | A09 | 38803185 |
| BN0529 | [C/T] | A09 | 39545280 |
| BN0530 | [C/T] | A09 | 40546328 |
| BN0531 | [A/C] | A09 | 40863518 |
| BN0532 | [T/A] | A09 | 42121681 |
| BN0533 | [G/A] | A09 | 43046674 |
| BN0534 | [A/C] | A09 | 43345325 |
| BN0535 | [G/A] | A09 | 43899902 |
| BN0536 | [T/A] | A09 | 44372059 |
| BN0537 | [C/A] | A09 | 44760067 |
| BN0538 | [G/C] | A09 | 45896819 |
| BN0539 | [G/A] | A09 | 46510941 |
| BN0540 | [C/A] | A09 | 47352882 |
| BN0541 | [C/A] | A09 | 47982192 |
| BN0542 | [C/T] | A09 | 48365495 |
| BN0543 | [A/T] | A09 | 48495780 |
| BN0544 | [C/T] | A09 | 48909687 |
| BN0545 | [C/T] | A09 | 49559700 |
| BN0546 | [G/T] | A09 | 49860591 |
| BN0547 | [G/A] | A09 | 50270440 |
| BN0548 | [G/C] | A09 | 51140947 |
| BN0549 | [A/G] | A09 | 51420252 |
| BN0550 | [T/G] | A09 | 51844532 |
| BN0551 | [C/T] | A09 | 52210525 |
| BN0552 | [G/C] | A09 | 52674285 |
| BN0553 | [T/C] | A09 | 53001318 |
| BN0554 | [T/G] | A10 | 293245 |
| BN0555 | [A/G] | A10 | 519105 |
| BN0556 | [G/T] | A10 | 1058320 |
| BN0557 | [C/G] | A10 | 1391954 |
| BN0558 | [G/T] | A10 | 1644622 |
| BN0559 | [A/G] | A10 | 1967264 |
| BN0560 | [A/C] | A10 | 2719788 |
| BN0561 | [A/G] | A10 | 3047568 |
| BN0562 | [G/T] | A10 | 3952229 |
| BN0563 | [T/G] | A10 | 4772203 |
| BN0564 | [T/C] | A10 | 4869005 |
| BN0565 | [G/A] | A10 | 6255054 |
| BN0566 | [C/T] | A10 | 6929260 |
| BN0567 | [C/T] | A10 | 7261047 |
| BN0568 | [A/C] | A10 | 9246270 |
| BN0569 | [A/G] | A10 | 10276067 |
| BN0570 | [A/G] | A10 | 10496200 |
| BN0571 | [C/T] | A10 | 11338128 |
| BN0572 | [G/C] | A10 | 11657997 |
| BN0573 | [G/A] | A10 | 12393777 |
| BN0574 | [T/G] | A10 | 12699587 |
| BN0575 | [A/G] | A10 | 13590381 |
| BN0576 | [T/C] | A10 | 13867538 |
| BN0577 | [A/T] | A10 | 14549628 |
| BN0578 | [C/T] | A10 | 15067397 |
| BN0579 | [C/G] | A10 | 15570898 |
| BN0580 | [G/A] | A10 | 15664032 |
| BN0581 | [A/G] | A10 | 16149149 |
| BN0582 | [T/A] | A10 | 16624529 |
| BN0583 | [A/T] | A10 | 17510683 |
| BN0584 | [C/A] | A10 | 18111756 |
| BN0585 | [T/A] | A10 | 18413345 |
| BN0586 | [G/A] | A10 | 18649419 |
| BN0587 | [G/A] | A10 | 19080114 |
| BN0588 | [G/T] | A10 | 19695808 |
| BN0589 | [A/G] | A10 | 19942920 |
| BN0590 | [T/A] | A10 | 20146695 |
| BN0591 | [G/C] | C01 | 8708 |
| BN0592 | [A/G] | C01 | 871836 |
| BN0593 | [G/A] | C01 | 1955455 |
| BN0594 | [G/A] | C01 | 2327745 |
| BN0595 | [T/C] | C01 | 2792164 |
| BN0596 | [C/T] | C01 | 3495216 |
| BN0597 | [T/C] | C01 | 4045803 |
| BN0598 | [T/C] | C01 | 4412983 |
| BN0599 | [C/T] | C01 | 4594315 |
| BN0600 | [A/C] | C01 | 5545134 |
| BN0601 | [C/T] | C01 | 5619624 |
| BN0602 | [A/G] | C01 | 6163796 |
| BN0603 | [G/A] | C01 | 6604131 |
| BN0604 | [C/T] | C01 | 7206428 |
| BN0605 | [G/A] | C01 | 7835279 |
| BN0606 | [C/A] | C01 | 9051193 |
| BN0607 | [G/T] | C01 | 9678225 |
| BN0608 | [T/A] | C01 | 9815380 |
| BN0609 | [T/A] | C01 | 10211368 |
| BN0610 | [A/G] | C01 | 10591847 |
| BN0611 | [T/A] | C01 | 10988051 |
| BN0612 | [A/C] | C01 | 11098406 |
| BN0613 | [C/A] | C01 | 12420842 |
| BN0614 | [C/A] | C01 | 12691362 |
| BN0615 | [G/A] | C01 | 13143732 |
| BN0616 | [G/C] | C01 | 13798685 |
| BN0617 | [G/A] | C01 | 14236494 |
| BN0618 | [C/T] | C01 | 14830021 |
| BN0619 | [G/T] | C01 | 16334917 |
| BN0620 | [C/T] | C01 | 17448440 |
| BN0621 | [T/G] | C01 | 17630184 |
| BN0622 | [C/T] | C01 | 18350592 |
| BN0623 | [T/C] | C01 | 19874920 |
| BN0624 | [A/C] | C01 | 20685434 |
| BN0625 | [G/C] | C01 | 22656715 |
| BN0626 | [T/A] | C01 | 23418558 |
| BN0627 | [A/G] | C01 | 23779523 |
| BN0628 | [A/C] | C01 | 25160006 |
| BN0629 | [G/C] | C01 | 26133860 |
| BN0630 | [A/G] | C01 | 26477005 |
| BN0631 | [T/G] | C01 | 27342159 |
| BN0632 | [A/G] | C01 | 29054098 |
| BN0633 | [A/G] | C01 | 29436093 |
| BN0634 | [C/T] | C01 | 29663679 |
| BN0635 | [A/C] | C01 | 30916636 |
| BN0636 | [C/T] | C01 | 31290776 |
| BN0637 | [A/C] | C01 | 31587341 |
| BN0638 | [T/A] | C01 | 32012992 |
| BN0639 | [T/G] | C01 | 33144223 |
| BN0640 | [C/T] | C01 | 33457842 |
| BN0641 | [A/G] | C01 | 33847873 |
| BN0642 | [A/G] | C01 | 34577660 |
| BN0643 | [G/A] | C01 | 35155759 |
| BN0644 | [T/G] | C01 | 35747007 |
| BN0645 | [C/A] | C01 | 36155019 |
| BN0646 | [T/G] | C01 | 37492587 |
| BN0647 | [C/A] | C01 | 37675108 |
| BN0648 | [C/T] | C01 | 38729557 |
| BN0649 | [C/G] | C01 | 39093335 |
| BN0650 | [T/A] | C01 | 39248157 |
| BN0651 | [T/G] | C01 | 39700878 |
| BN0652 | [G/C] | C01 | 40131785 |
| BN0653 | [A/T] | C01 | 40693595 |
| BN0654 | [T/C] | C01 | 40993969 |
| BN0655 | [T/C] | C01 | 41631061 |
| BN0656 | [C/A] | C01 | 42667054 |
| BN0657 | [C/T] | C01 | 43422067 |
| BN0658 | [G/A] | C01 | 43789928 |
| BN0659 | [G/T] | C01 | 44078953 |
| BN0660 | [C/A] | C01 | 44544380 |
| BN0661 | [A/T] | C01 | 45774141 |
| BN0662 | [C/T] | C01 | 46031495 |
| BN0663 | [C/A] | C01 | 46425279 |
| BN0664 | [C/T] | C02 | 446631 |
| BN0665 | [G/C] | C02 | 1433716 |
| BN0666 | [A/C] | C02 | 2060222 |
| BN0667 | [C/T] | C02 | 3596587 |
| BN0668 | [T/C] | C02 | 4259143 |
| BN0669 | [G/A] | C02 | 4512184 |
| BN0670 | [C/T] | C02 | 5848345 |
| BN0671 | [G/A] | C02 | 6211025 |
| BN0672 | [C/T] | C02 | 6957317 |
| BN0673 | [G/C] | C02 | 7624307 |
| BN0674 | [T/C] | C02 | 8071605 |
| BN0675 | [T/A] | C02 | 8657730 |
| BN0676 | [A/G] | C02 | 9881890 |
| BN0677 | [A/T] | C02 | 9923641 |
| BN0678 | [G/T] | C02 | 10916858 |
| BN0679 | [T/A] | C02 | 11841573 |
| BN0680 | [C/A] | C02 | 12212092 |
| BN0681 | [C/T] | C02 | 12920024 |
| BN0682 | [A/T] | C02 | 13367776 |
| BN0683 | [A/T] | C02 | 13880427 |
| BN0684 | [C/T] | C02 | 15053881 |
| BN0685 | [T/C] | C02 | 15721033 |
| BN0686 | [A/T] | C02 | 16250462 |
| BN0687 | [T/C] | C02 | 16643147 |
| BN0688 | [A/T] | C02 | 17185103 |
| BN0689 | [A/G] | C02 | 17335325 |
| BN0690 | [A/G] | C02 | 17962007 |
| BN0691 | [A/C] | C02 | 18098011 |
| BN0692 | [A/G] | C02 | 18448522 |
| BN0693 | [G/T] | C02 | 19699835 |
| BN0694 | [C/A] | C02 | 20122470 |
| BN0695 | [A/G] | C02 | 20845138 |
| BN0696 | [C/G] | C02 | 21605022 |
| BN0697 | [T/C] | C02 | 22020996 |
| BN0698 | [C/T] | C02 | 22606544 |
| BN0699 | [C/A] | C02 | 23891184 |
| BN0700 | [G/A] | C02 | 24787280 |
| BN0701 | [G/A] | C02 | 25343290 |
| BN0702 | [A/T] | C02 | 26083694 |
| BN0703 | [G/T] | C02 | 26868853 |
| BN0704 | [G/T] | C02 | 27791884 |
| BN0705 | [T/A] | C02 | 28011953 |
| BN0706 | [G/T] | C02 | 29339848 |
| BN0707 | [C/T] | C02 | 29648935 |
| BN0708 | [G/C] | C02 | 30508042 |
| BN0709 | [A/G] | C02 | 30715190 |
| BN0710 | [C/T] | C02 | 31020826 |
| BN0711 | [T/C] | C02 | 31619084 |
| BN0712 | [T/G] | C02 | 31904664 |
| BN0713 | [C/A] | C02 | 32248097 |
| BN0714 | [A/G] | C02 | 32792129 |
| BN0715 | [A/T] | C02 | 34119723 |
| BN0716 | [A/G] | C02 | 35276154 |
| BN0717 | [G/C] | C02 | 35598006 |
| BN0718 | [T/C] | C02 | 36417665 |
| BN0719 | [G/A] | C02 | 36884482 |
| BN0720 | [G/A] | C02 | 37368822 |
| BN0721 | [A/G] | C02 | 41069401 |
| BN0722 | [T/C] | C02 | 43238326 |
| BN0723 | [T/G] | C02 | 43410556 |
| BN0724 | [T/C] | C02 | 44514126 |
| BN0725 | [C/T] | C02 | 45350655 |
| BN0726 | [C/T] | C02 | 46392239 |
| BN0727 | [A/C] | C02 | 47919432 |
| BN0728 | [A/C] | C02 | 48597389 |
| BN0729 | [T/G] | C02 | 48852375 |
| BN0730 | [C/T] | C02 | 49082420 |
| BN0731 | [C/T] | C02 | 50134514 |
| BN0732 | [G/A] | C02 | 50485510 |
| BN0733 | [G/A] | C02 | 50803847 |
| BN0734 | [G/A] | C02 | 51625416 |
| BN0735 | [G/A] | C02 | 52894189 |
| BN0736 | [C/A] | C02 | 53525738 |
| BN0737 | [G/C] | C02 | 54975581 |
| BN0738 | [G/A] | C02 | 56138778 |
| BN0739 | [C/A] | C02 | 56845571 |
| BN0740 | [T/A] | C02 | 59077729 |
| BN0741 | [C/T] | C02 | 59209808 |
| BN0742 | [C/A] | C02 | 59861437 |
| BN0743 | [T/A] | C02 | 60085790 |
| BN0744 | [A/G] | C02 | 60820081 |
| BN0745 | [G/T] | C02 | 61293668 |
| BN0746 | [T/C] | C03 | 283272 |
| BN0747 | [T/C] | C03 | 793411 |
| BN0748 | [T/C] | C03 | 1191506 |
| BN0749 | [C/T] | C03 | 1893029 |
| BN0750 | [T/G] | C03 | 3356771 |
| BN0751 | [A/T] | C03 | 3722727 |
| BN0752 | [G/A] | C03 | 4186013 |
| BN0753 | [A/G] | C03 | 4448485 |
| BN0754 | [A/G] | C03 | 5233815 |
| BN0755 | [C/T] | C03 | 5635209 |
| BN0756 | [T/C] | C03 | 6329226 |
| BN0757 | [T/C] | C03 | 6444305 |
| BN0758 | [G/T] | C03 | 6774161 |
| BN0759 | [T/A] | C03 | 7023943 |
| BN0760 | [G/A] | C03 | 7143884 |
| BN0761 | [C/A] | C03 | 7836413 |
| BN0762 | [T/C] | C03 | 8039469 |
| BN0763 | [G/A] | C03 | 8562357 |
| BN0764 | [T/C] | C03 | 9045009 |
| BN0765 | [C/A] | C03 | 9703152 |
| BN0766 | [T/C] | C03 | 9824172 |
| BN0767 | [G/T] | C03 | 10129035 |
| BN0768 | [T/C] | C03 | 10456184 |
| BN0769 | [T/C] | C03 | 10700776 |
| BN0770 | [C/T] | C03 | 11520947 |
| BN0771 | [C/G] | C03 | 12034638 |
| BN0772 | [T/C] | C03 | 12552333 |
| BN0773 | [T/A] | C03 | 12839422 |
| BN0774 | [T/A] | C03 | 13118824 |
| BN0775 | [C/T] | C03 | 13530789 |
| BN0776 | [A/T] | C03 | 14031619 |
| BN0777 | [T/G] | C03 | 14705816 |
| BN0778 | [G/C] | C03 | 14847352 |
| BN0779 | [T/C] | C03 | 15203527 |
| BN0780 | [C/T] | C03 | 15994977 |
| BN0781 | [T/A] | C03 | 16450327 |
| BN0782 | [T/C] | C03 | 17083944 |
| BN0783 | [C/G] | C03 | 17369619 |
| BN0784 | [A/T] | C03 | 18032825 |
| BN0785 | [T/C] | C03 | 18175861 |
| BN0786 | [A/G] | C03 | 19844731 |
| BN0787 | [T/C] | C03 | 20218854 |
| BN0788 | [T/G] | C03 | 20794891 |
| BN0789 | [T/G] | C03 | 21553624 |
| BN0790 | [T/C] | C03 | 21944345 |
| BN0791 | [T/C] | C03 | 22811433 |
| BN0792 | [G/T] | C03 | 23174218 |
| BN0793 | [T/A] | C03 | 23359002 |
| BN0794 | [T/G] | C03 | 23687057 |
| BN0795 | [T/C] | C03 | 24134015 |
| BN0796 | [G/A] | C03 | 24343380 |
| BN0797 | [C/T] | C03 | 24850311 |
| BN0798 | [A/G] | C03 | 25926846 |
| BN0799 | [A/T] | C03 | 26092564 |
| BN0800 | [G/T] | C03 | 26723128 |
| BN0801 | [G/T] | C03 | 26966616 |
| BN0802 | [A/T] | C03 | 27278142 |
| BN0803 | [T/C] | C03 | 27715670 |
| BN0804 | [G/A] | C03 | 28006864 |
| BN0805 | [T/C] | C03 | 28330380 |
| BN0806 | [G/C] | C03 | 28864233 |
| BN0807 | [G/A] | C03 | 29358422 |
| BN0808 | [A/G] | C03 | 29980301 |
| BN0809 | [A/G] | C03 | 30716901 |
| BN0810 | [A/T] | C03 | 31057779 |
| BN0811 | [G/A] | C03 | 31222222 |
| BN0812 | [A/G] | C03 | 31541772 |
| BN0813 | [A/T] | C03 | 32730913 |
| BN0814 | [C/T] | C03 | 33797411 |
| BN0815 | [C/T] | C03 | 34348931 |
| BN0816 | [G/T] | C03 | 34716788 |
| BN0817 | [C/T] | C03 | 35240370 |
| BN0818 | [T/G] | C03 | 35435358 |
| BN0819 | [T/G] | C03 | 36462990 |
| BN0820 | [C/A] | C03 | 37305168 |
| BN0821 | [A/G] | C03 | 38328252 |
| BN0822 | [A/T] | C03 | 39685447 |
| BN0823 | [A/C] | C03 | 40174316 |
| BN0824 | [T/C] | C03 | 40436236 |
| BN0825 | [T/C] | C03 | 40832750 |
| BN0826 | [G/A] | C03 | 41174437 |
| BN0827 | [T/C] | C03 | 42037503 |
| BN0828 | [G/C] | C03 | 43143358 |
| BN0829 | [T/G] | C03 | 45682356 |
| BN0830 | [C/T] | C03 | 47803683 |
| BN0831 | [G/A] | C03 | 48176867 |
| BN0832 | [G/A] | C03 | 48469865 |
| BN0833 | [C/T] | C03 | 51281833 |
| BN0834 | [G/A] | C03 | 52595712 |
| BN0835 | [T/C] | C03 | 53389165 |
| BN0836 | [A/T] | C03 | 53847216 |
| BN0837 | [C/T] | C03 | 54244309 |
| BN0838 | [A/C] | C03 | 55381982 |
| BN0839 | [G/T] | C03 | 56045422 |
| BN0840 | [G/A] | C03 | 56387955 |
| BN0841 | [C/A] | C03 | 56865699 |
| BN0842 | [C/A] | C03 | 57546575 |
| BN0843 | [C/A] | C03 | 57741931 |
| BN0844 | [T/A] | C03 | 58349173 |
| BN0845 | [G/A] | C03 | 58604707 |
| BN0846 | [A/G] | C03 | 58750265 |
| BN0847 | [G/A] | C03 | 59077018 |
| BN0848 | [G/A] | C03 | 59355736 |
| BN0849 | [A/G] | C03 | 59623072 |
| BN0850 | [G/C] | C03 | 61088262 |
| BN0851 | [C/A] | C03 | 61373006 |
| BN0852 | [T/G] | C03 | 62013450 |
| BN0853 | [G/T] | C03 | 62383376 |
| BN0854 | [T/C] | C03 | 62705127 |
| BN0855 | [A/G] | C03 | 63221795 |
| BN0856 | [A/T] | C03 | 63590742 |
| BN0857 | [C/T] | C03 | 64089999 |
| BN0858 | [T/C] | C03 | 64990674 |
| BN0859 | [G/A] | C03 | 65877705 |
| BN0860 | [G/C] | C03 | 66817363 |
| BN0861 | [G/T] | C03 | 68192503 |
| BN0862 | [A/C] | C03 | 69462861 |
| BN0863 | [C/A] | C03 | 69840304 |
| BN0864 | [T/A] | C03 | 70158981 |
| BN0865 | [G/T] | C03 | 70578297 |
| BN0866 | [C/G] | C03 | 70782661 |
| BN0867 | [C/T] | C03 | 71054951 |
| BN0868 | [A/G] | C03 | 72375920 |
| BN0869 | [T/A] | C03 | 72867662 |
| BN0870 | [G/C] | C04 | 995760 |
| BN0871 | [C/T] | C04 | 1400602 |
| BN0872 | [A/G] | C04 | 1960851 |
| BN0873 | [A/G] | C04 | 2424972 |
| BN0874 | [G/A] | C04 | 4294617 |
| BN0875 | [T/C] | C04 | 5406538 |
| BN0876 | [T/C] | C04 | 6334157 |
| BN0877 | [G/A] | C04 | 6735716 |
| BN0878 | [C/T] | C04 | 7278952 |
| BN0879 | [T/C] | C04 | 7401501 |
| BN0880 | [A/G] | C04 | 8382077 |
| BN0881 | [A/G] | C04 | 8732463 |
| BN0882 | [T/A] | C04 | 9006183 |
| BN0883 | [T/C] | C04 | 9300205 |
| BN0884 | [T/C] | C04 | 9995799 |
| BN0885 | [G/C] | C04 | 10479058 |
| BN0886 | [A/C] | C04 | 10725573 |
| BN0887 | [A/G] | C04 | 11022285 |
| BN0888 | [G/A] | C04 | 11138648 |
| BN0889 | [C/T] | C04 | 12568524 |
| BN0890 | [G/T] | C04 | 13079880 |
| BN0891 | [C/G] | C04 | 13442727 |
| BN0892 | [G/T] | C04 | 13611098 |
| BN0893 | [G/T] | C04 | 14355281 |
| BN0894 | [T/C] | C04 | 15157327 |
| BN0895 | [T/G] | C04 | 16372862 |
| BN0896 | [A/G] | C04 | 17381862 |
| BN0897 | [T/G] | C04 | 17486079 |
| BN0898 | [A/G] | C04 | 18461994 |
| BN0899 | [A/G] | C04 | 19353982 |
| BN0900 | [C/T] | C04 | 19942430 |
| BN0901 | [T/C] | C04 | 20847446 |
| BN0902 | [A/G] | C04 | 21771065 |
| BN0903 | [G/T] | C04 | 22415089 |
| BN0904 | [T/A] | C04 | 23238753 |
| BN0905 | [G/C] | C04 | 24704796 |
| BN0906 | [G/A] | C04 | 27090952 |
| BN0907 | [G/T] | C04 | 27241498 |
| BN0908 | [A/G] | C04 | 27659190 |
| BN0909 | [C/G] | C04 | 28299122 |
| BN0910 | [C/T] | C04 | 29044180 |
| BN0911 | [C/A] | C04 | 29428417 |
| BN0912 | [T/C] | C04 | 29886413 |
| BN0913 | [C/A] | C04 | 31049038 |
| BN0914 | [G/T] | C04 | 31361167 |
| BN0915 | [C/T] | C04 | 31832393 |
| BN0916 | [C/G] | C04 | 32795738 |
| BN0917 | [T/G] | C04 | 32921459 |
| BN0918 | [A/G] | C04 | 33970759 |
| BN0919 | [A/G] | C04 | 34826469 |
| BN0920 | [A/C] | C04 | 35268016 |
| BN0921 | [T/C] | C04 | 35610048 |
| BN0922 | [G/A] | C04 | 35939884 |
| BN0923 | [T/C] | C04 | 36841052 |
| BN0924 | [G/C] | C04 | 37769811 |
| BN0925 | [G/A] | C04 | 38423726 |
| BN0926 | [G/A] | C04 | 38690510 |
| BN0927 | [G/A] | C04 | 39084985 |
| BN0928 | [T/C] | C04 | 39815017 |
| BN0929 | [G/T] | C04 | 40077317 |
| BN0930 | [C/T] | C04 | 40822742 |
| BN0931 | [C/T] | C04 | 41085145 |
| BN0932 | [A/T] | C04 | 41461884 |
| BN0933 | [G/A] | C04 | 41923499 |
| BN0934 | [G/C] | C04 | 42775603 |
| BN0935 | [C/A] | C04 | 43700308 |
| BN0936 | [A/G] | C04 | 44429632 |
| BN0937 | [T/A] | C04 | 45321143 |
| BN0938 | [A/G] | C04 | 45497566 |
| BN0939 | [A/T] | C04 | 46856810 |
| BN0940 | [G/A] | C04 | 48361577 |
| BN0941 | [A/C] | C04 | 48539855 |
| BN0942 | [T/C] | C04 | 48979973 |
| BN0943 | [G/A] | C04 | 49192842 |
| BN0944 | [T/C] | C04 | 50289946 |
| BN0945 | [A/C] | C04 | 51189585 |
| BN0946 | [G/A] | C04 | 51452289 |
| BN0947 | [A/T] | C04 | 51729695 |
| BN0948 | [T/C] | C04 | 52354747 |
| BN0949 | [A/G] | C04 | 52930373 |
| BN0950 | [G/A] | C04 | 53393153 |
| BN0951 | [T/C] | C04 | 54056106 |
| BN0952 | [C/T] | C04 | 54503099 |
| BN0953 | [G/A] | C04 | 55856030 |
| BN0954 | [T/C] | C04 | 56042753 |
| BN0955 | [A/T] | C04 | 56459789 |
| BN0956 | [G/T] | C04 | 56898406 |
| BN0957 | [T/G] | C04 | 58368239 |
| BN0958 | [C/A] | C04 | 58779466 |
| BN0959 | [G/A] | C04 | 59482111 |
| BN0960 | [A/G] | C04 | 60214734 |
| BN0961 | [C/T] | C04 | 60753969 |
| BN0962 | [G/C] | C04 | 61594114 |
| BN0963 | [G/T] | C04 | 61702147 |
| BN0964 | [A/G] | C04 | 62157069 |
| BN0965 | [A/G] | C04 | 62363676 |
| BN0966 | [G/T] | C04 | 62679888 |
| BN0967 | [T/C] | C04 | 63139920 |
| BN0968 | [G/A] | C04 | 63687680 |
| BN0969 | [C/T] | C05 | 565559 |
| BN0970 | [C/A] | C05 | 1075034 |
| BN0971 | [C/T] | C05 | 1333060 |
| BN0972 | [C/A] | C05 | 1702479 |
| BN0973 | [G/T] | C05 | 1823004 |
| BN0974 | [T/G] | C05 | 2549850 |
| BN0975 | [G/C] | C05 | 3167714 |
| BN0976 | [C/A] | C05 | 3376130 |
| BN0977 | [G/C] | C05 | 4027941 |
| BN0978 | [G/A] | C05 | 5340419 |
| BN0979 | [C/T] | C05 | 5428513 |
| BN0980 | [C/T] | C05 | 5763702 |
| BN0981 | [A/G] | C05 | 5949947 |
| BN0982 | [T/C] | C05 | 6465242 |
| BN0983 | [C/T] | C05 | 7266432 |
| BN0984 | [C/T] | C05 | 7363682 |
| BN0985 | [G/A] | C05 | 7864864 |
| BN0986 | [T/C] | C05 | 7999522 |
| BN0987 | [C/G] | C05 | 8593851 |
| BN0988 | [T/C] | C05 | 8704056 |
| BN0989 | [C/T] | C05 | 8983821 |
| BN0990 | [T/A] | C05 | 10554193 |
| BN0991 | [A/C] | C05 | 11519749 |
| BN0992 | [A/T] | C05 | 12407458 |
| BN0993 | [C/A] | C05 | 12715647 |
| BN0994 | [C/T] | C05 | 12856641 |
| BN0995 | [C/A] | C05 | 14204463 |
| BN0996 | [C/T] | C05 | 14436369 |
| BN0997 | [A/G] | C05 | 15555371 |
| BN0998 | [C/A] | C05 | 15647726 |
| BN0999 | [A/G] | C05 | 16415555 |
| BN1000 | [G/T] | C05 | 17280802 |
| BN1001 | [A/T] | C05 | 18378861 |
| BN1002 | [G/A] | C05 | 18799222 |
| BN1003 | [A/G] | C05 | 18928599 |
| BN1004 | [A/G] | C05 | 19305763 |
| BN1005 | [A/G] | C05 | 19844481 |
| BN1006 | [A/T] | C05 | 20006835 |
| BN1007 | [C/T] | C05 | 20233529 |
| BN1008 | [G/C] | C05 | 21964136 |
| BN1009 | [C/T] | C05 | 22739956 |
| BN1010 | [C/T] | C05 | 22900999 |
| BN1011 | [C/T] | C05 | 23262361 |
| BN1012 | [A/G] | C05 | 24308126 |
| BN1013 | [G/A] | C05 | 25454587 |
| BN1014 | [C/A] | C05 | 26182201 |
| BN1015 | [C/G] | C05 | 26467598 |
| BN1016 | [C/G] | C05 | 27248026 |
| BN1017 | [C/T] | C05 | 27454601 |
| BN1018 | [G/A] | C05 | 28549353 |
| BN1019 | [A/T] | C05 | 29074087 |
| BN1020 | [A/G] | C05 | 29228647 |
| BN1021 | [G/A] | C05 | 29561576 |
| BN1022 | [A/C] | C05 | 29932394 |
| BN1023 | [A/G] | C05 | 32145906 |
| BN1024 | [G/A] | C05 | 32727296 |
| BN1025 | [A/C] | C05 | 32985417 |
| BN1026 | [G/T] | C05 | 33453957 |
| BN1027 | [A/G] | C05 | 34035803 |
| BN1028 | [C/T] | C05 | 34507047 |
| BN1029 | [T/A] | C05 | 34699231 |
| BN1030 | [T/C] | C05 | 35100756 |
| BN1031 | [A/T] | C05 | 35316632 |
| BN1032 | [C/G] | C05 | 35839965 |
| BN1033 | [T/G] | C05 | 36414172 |
| BN1034 | [A/G] | C05 | 36808715 |
| BN1035 | [C/T] | C05 | 37808693 |
| BN1036 | [C/T] | C05 | 38346379 |
| BN1037 | [G/A] | C05 | 39074064 |
| BN1038 | [C/T] | C05 | 39954408 |
| BN1039 | [A/G] | C05 | 41699687 |
| BN1040 | [T/C] | C05 | 42739412 |
| BN1041 | [G/T] | C05 | 43232743 |
| BN1042 | [C/A] | C05 | 43425261 |
| BN1043 | [T/C] | C05 | 44034668 |
| BN1044 | [T/A] | C05 | 44664713 |
| BN1045 | [C/A] | C05 | 45027392 |
| BN1046 | [C/T] | C05 | 45901215 |
| BN1047 | [C/G] | C05 | 46352592 |
| BN1048 | [T/A] | C05 | 46708275 |
| BN1049 | [C/A] | C05 | 46941796 |
| BN1050 | [C/G] | C05 | 47312938 |
| BN1051 | [G/A] | C05 | 49395415 |
| BN1052 | [A/T] | C05 | 50856718 |
| BN1053 | [T/A] | C05 | 51096431 |
| BN1054 | [A/G] | C05 | 51492585 |
| BN1055 | [C/G] | C05 | 52173378 |
| BN1056 | [C/T] | C05 | 54006722 |
| BN1057 | [C/A] | C05 | 54311819 |
| BN1058 | [A/G] | C05 | 54906161 |
| BN1059 | [G/A] | C06 | 4295963 |
| BN1060 | [A/T] | C06 | 4495052 |
| BN1061 | [G/A] | C06 | 5451146 |
| BN1062 | [A/T] | C06 | 6262483 |
| BN1063 | [A/C] | C06 | 6650602 |
| BN1064 | [G/A] | C06 | 7093445 |
| BN1065 | [C/A] | C06 | 7269980 |
| BN1066 | [G/T] | C06 | 7922352 |
| BN1067 | [C/A] | C06 | 8388494 |
| BN1068 | [G/T] | C06 | 8627166 |
| BN1069 | [G/T] | C06 | 9058908 |
| BN1070 | [A/G] | C06 | 9223161 |
| BN1071 | [G/A] | C06 | 9536029 |
| BN1072 | [T/C] | C06 | 10650709 |
| BN1073 | [C/A] | C06 | 11131970 |
| BN1074 | [C/T] | C06 | 11517831 |
| BN1075 | [G/T] | C06 | 11623464 |
| BN1076 | [A/G] | C06 | 12321749 |
| BN1077 | [G/T] | C06 | 13034901 |
| BN1078 | [G/A] | C06 | 13574003 |
| BN1079 | [T/C] | C06 | 14305562 |
| BN1080 | [C/T] | C06 | 14415859 |
| BN1081 | [A/C] | C06 | 15277464 |
| BN1082 | [G/A] | C06 | 15456411 |
| BN1083 | [T/G] | C06 | 15703128 |
| BN1084 | [C/T] | C06 | 15948506 |
| BN1085 | [G/A] | C06 | 16335997 |
| BN1086 | [C/G] | C06 | 17750558 |
| BN1087 | [C/T] | C06 | 17899659 |
| BN1088 | [C/T] | C06 | 18661395 |
| BN1089 | [A/T] | C06 | 19635730 |
| BN1090 | [T/G] | C06 | 20034183 |
| BN1091 | [C/T] | C06 | 20502253 |
| BN1092 | [C/T] | C06 | 22029339 |
| BN1093 | [A/G] | C06 | 22916161 |
| BN1094 | [T/C] | C06 | 23399985 |
| BN1095 | [A/T] | C06 | 23873225 |
| BN1096 | [A/G] | C06 | 24073443 |
| BN1097 | [T/A] | C06 | 24333233 |
| BN1098 | [C/T] | C06 | 24871829 |
| BN1099 | [C/A] | C06 | 25258827 |
| BN1100 | [A/G] | C06 | 25832607 |
| BN1101 | [C/T] | C06 | 26544808 |
| BN1102 | [G/A] | C06 | 26886927 |
| BN1103 | [C/T] | C06 | 27073498 |
| BN1104 | [T/C] | C06 | 27252149 |
| BN1105 | [C/G] | C06 | 28582234 |
| BN1106 | [C/T] | C06 | 29152897 |
| BN1107 | [C/G] | C06 | 29696118 |
| BN1108 | [G/A] | C06 | 29888627 |
| BN1109 | [G/T] | C06 | 30178506 |
| BN1110 | [A/G] | C06 | 30760973 |
| BN1111 | [G/A] | C06 | 31149536 |
| BN1112 | [T/G] | C06 | 31344860 |
| BN1113 | [C/T] | C06 | 31943521 |
| BN1114 | [T/C] | C06 | 32237886 |
| BN1115 | [A/C] | C06 | 32526730 |
| BN1116 | [G/A] | C06 | 32837641 |
| BN1117 | [T/A] | C06 | 33042448 |
| BN1118 | [T/C] | C06 | 33170630 |
| BN1119 | [G/C] | C06 | 33763816 |
| BN1120 | [G/C] | C06 | 35104213 |
| BN1121 | [G/C] | C06 | 35538572 |
| BN1122 | [A/C] | C06 | 35895789 |
| BN1123 | [T/C] | C06 | 36670077 |
| BN1124 | [A/G] | C06 | 36912105 |
| BN1125 | [G/A] | C06 | 37581336 |
| BN1126 | [A/G] | C06 | 37810453 |
| BN1127 | [T/G] | C06 | 38465249 |
| BN1128 | [G/A] | C06 | 38511501 |
| BN1129 | [C/T] | C06 | 40176880 |
| BN1130 | [A/G] | C06 | 40480463 |
| BN1131 | [T/C] | C06 | 41422906 |
| BN1132 | [T/C] | C06 | 41534728 |
| BN1133 | [C/T] | C06 | 42057537 |
| BN1134 | [T/G] | C06 | 42606103 |
| BN1135 | [T/A] | C06 | 42857680 |
| BN1136 | [A/T] | C06 | 45113731 |
| BN1137 | [T/C] | C06 | 45404469 |
| BN1138 | [G/A] | C06 | 45730301 |
| BN1139 | [A/T] | C06 | 46704746 |
| BN1140 | [G/T] | C06 | 46983810 |
| BN1141 | [T/G] | C06 | 47793183 |
| BN1142 | [T/A] | C06 | 48421426 |
| BN1143 | [T/A] | C06 | 48958810 |
| BN1144 | [T/A] | C06 | 49307537 |
| BN1145 | [A/G] | C06 | 49418342 |
| BN1146 | [A/C] | C06 | 49821766 |
| BN1147 | [G/A] | C06 | 50004479 |
| BN1148 | [T/A] | C07 | 3185168 |
| BN1149 | [T/C] | C07 | 3462520 |
| BN1150 | [C/A] | C07 | 3719282 |
| BN1151 | [C/T] | C07 | 5137118 |
| BN1152 | [C/T] | C07 | 6774856 |
| BN1153 | [T/A] | C07 | 7150922 |
| BN1154 | [C/T] | C07 | 7594885 |
| BN1155 | [G/C] | C07 | 7897356 |
| BN1156 | [T/G] | C07 | 9401404 |
| BN1157 | [T/C] | C07 | 9506508 |
| BN1158 | [G/A] | C07 | 9729125 |
| BN1159 | [T/G] | C07 | 10294157 |
| BN1160 | [A/G] | C07 | 11242970 |
| BN1161 | [T/C] | C07 | 12672736 |
| BN1162 | [A/T] | C07 | 13729250 |
| BN1163 | [A/C] | C07 | 13857620 |
| BN1164 | [A/C] | C07 | 14460382 |
| BN1165 | [A/G] | C07 | 14603257 |
| BN1166 | [T/C] | C07 | 14923357 |
| BN1167 | [G/A] | C07 | 15260645 |
| BN1168 | [T/C] | C07 | 15683443 |
| BN1169 | [T/C] | C07 | 16508903 |
| BN1170 | [C/G] | C07 | 17091508 |
| BN1171 | [A/G] | C07 | 17530607 |
| BN1172 | [G/A] | C07 | 17985851 |
| BN1173 | [G/A] | C07 | 20940241 |
| BN1174 | [A/G] | C07 | 21185852 |
| BN1175 | [C/G] | C07 | 22530834 |
| BN1176 | [G/C] | C07 | 22757098 |
| BN1177 | [T/A] | C07 | 23602300 |
| BN1178 | [A/G] | C07 | 23767891 |
| BN1179 | [T/C] | C07 | 24113860 |
| BN1180 | [G/T] | C07 | 24364883 |
| BN1181 | [G/A] | C07 | 24472900 |
| BN1182 | [A/G] | C07 | 25085563 |
| BN1183 | [C/T] | C07 | 25580594 |
| BN1184 | [C/T] | C07 | 25995417 |
| BN1185 | [G/A] | C07 | 26147121 |
| BN1186 | [G/A] | C07 | 27694932 |
| BN1187 | [G/A] | C07 | 28040643 |
| BN1188 | [A/G] | C07 | 28767945 |
| BN1189 | [A/G] | C07 | 29073845 |
| BN1190 | [T/C] | C07 | 30029991 |
| BN1191 | [G/A] | C07 | 30705429 |
| BN1192 | [G/A] | C07 | 30947973 |
| BN1193 | [G/A] | C07 | 33513639 |
| BN1194 | [C/T] | C07 | 33621738 |
| BN1195 | [A/T] | C07 | 33988954 |
| BN1196 | [T/A] | C07 | 34326014 |
| BN1197 | [A/T] | C07 | 34684874 |
| BN1198 | [G/A] | C07 | 35023844 |
| BN1199 | [T/C] | C07 | 35229659 |
| BN1200 | [G/A] | C07 | 35670088 |
| BN1201 | [T/C] | C07 | 36617434 |
| BN1202 | [G/C] | C07 | 37313522 |
| BN1203 | [G/T] | C07 | 37535129 |
| BN1204 | [A/G] | C07 | 37759015 |
| BN1205 | [T/C] | C07 | 37935449 |
| BN1206 | [T/C] | C07 | 38473476 |
| BN1207 | [T/A] | C07 | 39355257 |
| BN1208 | [T/G] | C07 | 39744518 |
| BN1209 | [T/C] | C07 | 40863893 |
| BN1210 | [G/A] | C07 | 41326168 |
| BN1211 | [A/G] | C07 | 41673325 |
| BN1212 | [G/A] | C07 | 42388350 |
| BN1213 | [A/C] | C07 | 42591585 |
| BN1214 | [G/A] | C07 | 42907629 |
| BN1215 | [A/C] | C07 | 43183210 |
| BN1216 | [T/C] | C07 | 44040636 |
| BN1217 | [C/T] | C07 | 44224040 |
| BN1218 | [A/G] | C07 | 45160601 |
| BN1219 | [C/T] | C07 | 46953708 |
| BN1220 | [T/A] | C07 | 47928176 |
| BN1221 | [T/C] | C07 | 48555252 |
| BN1222 | [T/C] | C07 | 48754107 |
| BN1223 | [T/C] | C07 | 49153780 |
| BN1224 | [T/A] | C07 | 49608749 |
| BN1225 | [C/T] | C07 | 49827371 |
| BN1226 | [G/A] | C07 | 50030300 |
| BN1227 | [G/A] | C07 | 50580398 |
| BN1228 | [G/T] | C07 | 51101157 |
| BN1229 | [T/C] | C07 | 51215560 |
| BN1230 | [A/T] | C07 | 51820234 |
| BN1231 | [C/T] | C07 | 52183821 |
| BN1232 | [G/A] | C07 | 52832516 |
| BN1233 | [T/C] | C07 | 53819867 |
| BN1234 | [G/A] | C07 | 54220755 |
| BN1235 | [C/T] | C08 | 177880 |
| BN1236 | [T/A] | C08 | 388338 |
| BN1237 | [C/T] | C08 | 884352 |
| BN1238 | [A/T] | C08 | 1716856 |
| BN1239 | [G/A] | C08 | 2184665 |
| BN1240 | [T/C] | C08 | 2703255 |
| BN1241 | [G/A] | C08 | 2908013 |
| BN1242 | [A/G] | C08 | 3306204 |
| BN1243 | [T/C] | C08 | 3692903 |
| BN1244 | [A/G] | C08 | 4433900 |
| BN1245 | [T/C] | C08 | 5611113 |
| BN1246 | [T/C] | C08 | 6442533 |
| BN1247 | [G/A] | C08 | 6876605 |
| BN1248 | [T/A] | C08 | 7266091 |
| BN1249 | [T/C] | C08 | 8554852 |
| BN1250 | [C/T] | C08 | 8936830 |
| BN1251 | [T/A] | C08 | 11435202 |
| BN1252 | [T/A] | C08 | 14430095 |
| BN1253 | [C/T] | C08 | 14875646 |
| BN1254 | [A/C] | C08 | 15002413 |
| BN1255 | [T/C] | C08 | 15321671 |
| BN1256 | [G/C] | C08 | 15973314 |
| BN1257 | [G/C] | C08 | 17489863 |
| BN1258 | [G/A] | C08 | 18326046 |
| BN1259 | [G/A] | C08 | 18593854 |
| BN1260 | [C/T] | C08 | 18899610 |
| BN1261 | [G/A] | C08 | 19194404 |
| BN1262 | [G/A] | C08 | 19362263 |
| BN1263 | [G/A] | C08 | 19750124 |
| BN1264 | [A/C] | C08 | 20445933 |
| BN1265 | [C/G] | C08 | 21861820 |
| BN1266 | [G/T] | C08 | 22323771 |
| BN1267 | [G/T] | C08 | 24160742 |
| BN1268 | [C/T] | C08 | 24588948 |
| BN1269 | [A/T] | C08 | 25200160 |
| BN1270 | [C/T] | C08 | 25845594 |
| BN1271 | [A/G] | C08 | 25926150 |
| BN1272 | [C/T] | C08 | 26600972 |
| BN1273 | [T/C] | C08 | 27905734 |
| BN1274 | [C/A] | C08 | 28136954 |
| BN1275 | [T/A] | C08 | 28597508 |
| BN1276 | [G/A] | C08 | 29453028 |
| BN1277 | [G/A] | C08 | 30241749 |
| BN1278 | [A/G] | C08 | 30803076 |
| BN1279 | [G/A] | C08 | 30971031 |
| BN1280 | [A/G] | C08 | 31378887 |
| BN1281 | [G/A] | C08 | 31506095 |
| BN1282 | [T/G] | C08 | 31810252 |
| BN1283 | [G/A] | C08 | 32088223 |
| BN1284 | [A/G] | C08 | 32468571 |
| BN1285 | [C/T] | C08 | 32764069 |
| BN1286 | [G/A] | C08 | 33052855 |
| BN1287 | [A/C] | C08 | 33403297 |
| BN1288 | [T/A] | C08 | 35063901 |
| BN1289 | [C/G] | C08 | 36358337 |
| BN1290 | [G/T] | C08 | 36858692 |
| BN1291 | [A/C] | C08 | 37570513 |
| BN1292 | [C/T] | C08 | 37733876 |
| BN1293 | [C/T] | C08 | 38233207 |
| BN1294 | [C/T] | C08 | 38600698 |
| BN1295 | [C/G] | C08 | 39286345 |
| BN1296 | [C/A] | C08 | 39451976 |
| BN1297 | [C/T] | C08 | 39805862 |
| BN1298 | [G/A] | C08 | 40844780 |
| BN1299 | [T/C] | C09 | 1283762 |
| BN1300 | [C/T] | C09 | 2304641 |
| BN1301 | [A/G] | C09 | 2830555 |
| BN1302 | [A/C] | C09 | 3377206 |
| BN1303 | [G/A] | C09 | 3777259 |
| BN1304 | [A/G] | C09 | 4134282 |
| BN1305 | [T/C] | C09 | 4333788 |
| BN1306 | [G/A] | C09 | 4533109 |
| BN1307 | [C/T] | C09 | 4808041 |
| BN1308 | [G/A] | C09 | 5044005 |
| BN1309 | [C/A] | C09 | 5381471 |
| BN1310 | [C/T] | C09 | 6005186 |
| BN1311 | [G/A] | C09 | 6269584 |
| BN1312 | [C/T] | C09 | 7027094 |
| BN1313 | [C/T] | C09 | 7408170 |
| BN1314 | [C/T] | C09 | 7669880 |
| BN1315 | [A/G] | C09 | 8216737 |
| BN1316 | [A/G] | C09 | 8619683 |
| BN1317 | [T/C] | C09 | 9290670 |
| BN1318 | [G/A] | C09 | 9942436 |
| BN1319 | [C/T] | C09 | 10504604 |
| BN1320 | [C/T] | C09 | 10790093 |
| BN1321 | [G/T] | C09 | 11934178 |
| BN1322 | [A/T] | C09 | 12329017 |
| BN1323 | [G/T] | C09 | 12823226 |
| BN1324 | [G/A] | C09 | 13798833 |
| BN1325 | [C/T] | C09 | 14121467 |
| BN1326 | [T/C] | C09 | 14963317 |
| BN1327 | [A/G] | C09 | 15984783 |
| BN1328 | [C/T] | C09 | 17276337 |
| BN1329 | [G/A] | C09 | 17680228 |
| BN1330 | [C/A] | C09 | 18275408 |
| BN1331 | [G/A] | C09 | 18911713 |
| BN1332 | [G/C] | C09 | 19038837 |
| BN1333 | [T/C] | C09 | 19789379 |
| BN1334 | [A/G] | C09 | 20806485 |
| BN1335 | [T/A] | C09 | 21038568 |
| BN1336 | [A/C] | C09 | 21738410 |
| BN1337 | [A/G] | C09 | 22631400 |
| BN1338 | [G/A] | C09 | 22992740 |
| BN1339 | [G/A] | C09 | 24713578 |
| BN1340 | [T/A] | C09 | 25008931 |
| BN1341 | [A/T] | C09 | 25933263 |
| BN1342 | [C/T] | C09 | 26073109 |
| BN1343 | [G/A] | C09 | 26639966 |
| BN1344 | [G/A] | C09 | 26798967 |
| BN1345 | [G/T] | C09 | 28153609 |
| BN1346 | [G/A] | C09 | 28567797 |
| BN1347 | [T/A] | C09 | 29423970 |
| BN1348 | [A/G] | C09 | 29958682 |
| BN1349 | [C/T] | C09 | 30417856 |
| BN1350 | [T/C] | C09 | 31953801 |
| BN1351 | [C/T] | C09 | 32362479 |
| BN1352 | [T/G] | C09 | 33418713 |
| BN1353 | [G/C] | C09 | 33730552 |
| BN1354 | [C/T] | C09 | 36827877 |
| BN1355 | [A/C] | C09 | 38232699 |
| BN1356 | [T/A] | C09 | 39038814 |
| BN1357 | [T/C] | C09 | 40088426 |
| BN1358 | [A/C] | C09 | 40460087 |
| BN1359 | [T/G] | C09 | 41112813 |
| BN1360 | [C/T] | C09 | 41727885 |
| BN1361 | [G/A] | C09 | 42941490 |
| BN1362 | [G/C] | C09 | 43260317 |
| BN1363 | [A/G] | C09 | 44878151 |
| BN1364 | [A/G] | C09 | 45113275 |
| BN1365 | [G/A] | C09 | 46417761 |
| BN1366 | [T/C] | C09 | 46837315 |
| BN1367 | [C/T] | C09 | 47819202 |
| BN1368 | [T/C] | C09 | 48188055 |
| BN1369 | [A/G] | C09 | 48641036 |
| BN1370 | [G/A] | C09 | 48759717 |
| BN1371 | [G/C] | C09 | 49618393 |
| BN1372 | [A/G] | C09 | 50012536 |
| BN1373 | [G/A] | C09 | 50506492 |
| BN1374 | [C/T] | C09 | 50679051 |
| BN1375 | [C/A] | C09 | 51197804 |
| BN1376 | [A/G] | C09 | 52440542 |
| BN1377 | [T/G] | C09 | 52531485 |
| BN1378 | [G/A] | C09 | 52913562 |
| BN1379 | [T/G] | C09 | 53310301 |
| BN1380 | [T/C] | C09 | 53445902 |
| BN1381 | [A/G] | C09 | 54025969 |
| BN1382 | [T/A] | C09 | 55256069 |
| BN1383 | [G/A] | C09 | 56521841 |
| BN1384 | [A/G] | C09 | 57356786 |
| BN1385 | [T/C] | C09 | 57484709 |
| BN1386 | [A/G] | C09 | 57843296 |
| BN1387 | [A/G] | C09 | 58297949 |
| BN1388 | [C/A] | C09 | 58697511 |
| BN1389 | [A/G] | C09 | 59155815 |
| BN1390 | [G/A] | C09 | 59446910 |
| BN1391 | [A/T] | C09 | 60000799 |
| BN1392 | [A/T] | C09 | 60339769 |
| BN1393 | [C/T] | C09 | 60771136 |
| BN1394 | [T/A] | C09 | 61053345 |
| BN1395 | [G/A] | C09 | 61178632 |
| BN1396 | [A/G] | C09 | 61577411 |
| BN1397 | [C/T] | C09 | 62297148 |
| BN1398 | [C/T] | C09 | 62741728 |
| BN1399 | [C/G] | C09 | 63020374 |
| BN1400 | [G/T] | C09 | 63171947 |
| BN1401 | [A/C] | C09 | 63330083 |
| BN1402 | [A/G] | C09 | 63522057 |
| BN1403 | [T/A] | C09 | 63986029 |
| BN1404 | [A/G] | C09 | 64214135 |
| BN1405 | [T/C] | C09 | 64901846 |
| BN1406 | [A/T] | C09 | 65140654 |
| BN1407 | [G/A] | C09 | 66302905 |

It should be understood that, taking the maker BN0001 as an example, the nucleotide on the SNP site at the physical position 129037 of the chromosome A01 can be adenine (A) or guanine (G).

According to a second aspect, the disclosure proposes an SNP chip of Brassica napus L., and the SNP chip of Brassica napus L. includes at least one of a probe and a primer for detecting the SNP molecular marker combination of Brassica napus L.

In some embodiments of the disclosure, the SNP chip includes a liquid-phase probe for detecting the SNP molecular marker combination of Brassica napus L. Standardized automated detection and analysis can be easily achieved by preparing the SNP chip, and combining a targeted genotyping technology based on liquid-phase probe hybridization.

According to a third aspect, the disclosure proposes a kit, and the kit includes at least one of a probe and a primer for detecting the SNP molecular marker combination of Brassica napus L.

According to a fourth aspect, the disclosure proposes an application method of the SNP molecular marker combination of Brassica napus L., the kit, or the SNP chip of Brassica napus L., and the application method includes: genotyping the Brassica napus L. by using the SNP molecular marker combination, the kit, or the SNP chip.

In some embodiments of the disclosure, the application method includes: performing genome-wide association study on the Brassica napus L. by using the SNP molecular marker combination, the kit, or the SNP chip.

In some embodiments of the disclosure, the application method includes: performing cluster analysis on the Brassica napus L. by using the SNP molecular marker combination, the kit, or the SNP chip.

In some embodiments of the disclosure, the application method includes: performing paternity testing on the Brassica napus L. by using the SNP molecular marker combination, the kit, or the SNP chip.

In some embodiments of the disclosure, the application method includes: performing one of breeding or assisted breeding on the Brassica napus L. by using the SNP molecular marker combination, the kit, or the SNP chip.

In some embodiments of the disclosure, the one of breeding or assisted breeding includes at least one selected from the group consisting of assisted major gene selection, backcrossing breeding and background selection, genomic selection breeding, molecular-assisted breeding, seed purity testing, genetically modified component identification, quantitative trait loci (QTL) analysis, species evolution analysis and germplasm resource identification.

A breeding method of Brassica napus L. is provided, and the method includes: detecting deoxyribonucleic acid (DNA) of to-be-detected Brassica napus L. by using the aforementioned SNP molecular marker combination, the kit, or the SNP chip, and selecting a target Brassica napus L. for subsequent breeding.

In some embodiments of the disclosure, the detection is performed based on multiplex polymerase chain reaction (PCR) and sequencing.

According to some embodiments of the disclosure, the disclosure at least have the following beneficial effects. The disclosure screens a set of 1407 high-quality and highly polymorphic SNP molecular markers (minor allele frequency abbreviated as MAF value of the 1407 SNP molecular markers in 354 varieties of Brassica napus L. are greater than or equal to 0.15), and the 1407 SNP molecular markers are co-dominant markers, which have high specificity, sensitivity and resolution, and have wide application universality. The 1407 SNP molecular markers are not affected by environmental conditions and can be detected in by using seeds or any type of plant tissue. The detection results are accurate and have good repeatability and stability. The different data results obtained from different testing laboratories can be compared and verified with each other, and the data has universal comparability. A 1.5 K (i.e., 1500) SNP chip is prepared by using the 1407 SNP molecular markers, so as to achieve the detection of genome-wide SNP of Brassica napus L. samples quickly, with high-throughput and low-cost, which can be effectively used for breeding or assisted breeding of Brassica napus L.

### BRIEF DESCRIPTION OF DRAWINGS

The disclosure will be further described in conjunction with drawings and embodiments below.

FIG. 1 illustrates a flowchart of locus development according to an embodiment 1 of the disclosure.

FIG. 2 illustrates a distribution diagram of SNP loci in chromosomes of Brassica napus L. according to the embodiment 1 of the disclosure.

FIG. 3 illustrates a detection flowchart of Brassica napus L. samples based on multiplex PCR and next generation sequencing (also referred to as second generation sequencing) according to an embodiment 2 of the disclosure.

FIG. 4 illustrates a schematic diagram of cluster analysis according to the embodiment 2 of the disclosure.

FIG. 5 illustrates a schematic diagram of a phylogenetic tree of Brassica napus L. samples according to the embodiment 2 of the disclosure.

FIG. 6 illustrates a schematic diagram of a difference result in the chromosomes of Brassica napus L. according to an embodiment 3 of the disclosure.

### DETAILED DESCRIPTION OF EMBODIMENTS

Conceptions and technical effects of the disclosure are clearly and completely described in conjunction with embodiments below, to fully understand a purpose, features and effects of the disclosure. Apparently, the described embodiments are some of the embodiments of the disclosure, not all of them. For those skilled in the art, other embodiments obtained based on the embodiments of the disclosure without creative work fall within a scope of protection of the disclosure.

### Embodiment 1

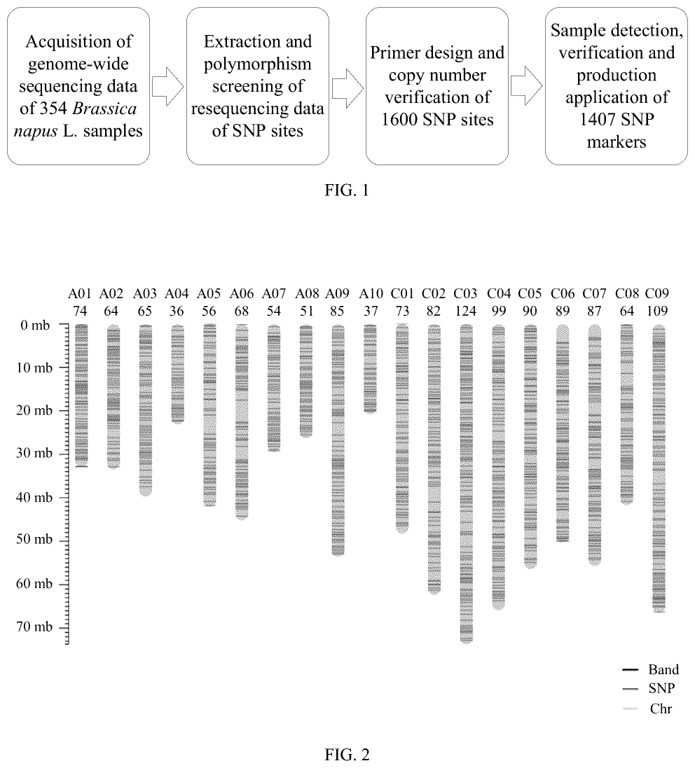

The embodiment provides a SNP molecular marker combination of Brassica napus L., and the SNP molecular marker combination includes 1407 SNP molecular markers. Each SNP molecular marker includes two different base mutation loci, and the base mutation loci are used to detect allele change of the SNP molecular marker. Physical positions of the 1407 SNP molecular markers are determined by sequence alignment based on a reference genome (Brana_Dar_V10) of Brassica napus L., and the 1407 SNP molecular markers are shown in BN0001-1407 of summary (specific information is shown in Table BN0001-1407). A screening process of the 1407 SNP molecular markers is shown in FIG. 1, and is specifically described as follows.

1. Acquisition of Germplasm Resource Data of Brassica napus L.

276 core germplasm materials of Brassica napus L. of Zhejiang university, China, and 78 core germplasm materials of Brassica napus L. in Hunan province, China, are used to extract resequencing data to together form a germplasm resource database of Brassica napus L.

2. Extraction and Screening of SNP Loci of Brassica napus L.

The obtained data of 354 core germplasm resources of Brassica napus L. is used to extract SNP loci, and a polymorphism analysis is performed on the extracted SNP loci to obtain 53584 SNP loci. The collected 53584 SNP loci are aligned with the reference genome (Brana_Dar_V10) of Brassica napus L. through selecting a locus interval about 500 K base pairs (bp) to obtain 2088 well matched SNP loci.

3. Synthesis and Selection of 1600 Genome-Wide Markers of Brassica napus L.

The sequence alignment is further performed on the 2088 SNP loci and the reference genome (Brana_Dar_V10) of Brassica napus L. to obtain 1600 SNP loci with optimal specificity. Primer design and copy number analysis are performed on the 1600 SNP loci. In addition, 78 Brassica napus L. materials are selected for genotyping validation. From this process, 1407 SNP molecular markers with high polymorphism and a data detection rate greater than 97% are carefully selected, which are suitable for genome-wide SNP detection in the Brassica napus L. samples. The distribution diagram of the SNP loci of the disclosure in the Brassica napus L. chromosomes is shown in FIG. 2.

The obtained 1407 SNP loci are used as a basis to design a primer and prepare a 1.5 K genome-wide liquid-phase gene chip for detecting the 1407 SNP loci.

### Embodiment 2

The embodiment provides an application method of the SNP molecular marker combination in cluster analysis and paternity testing of Brassica napus L.

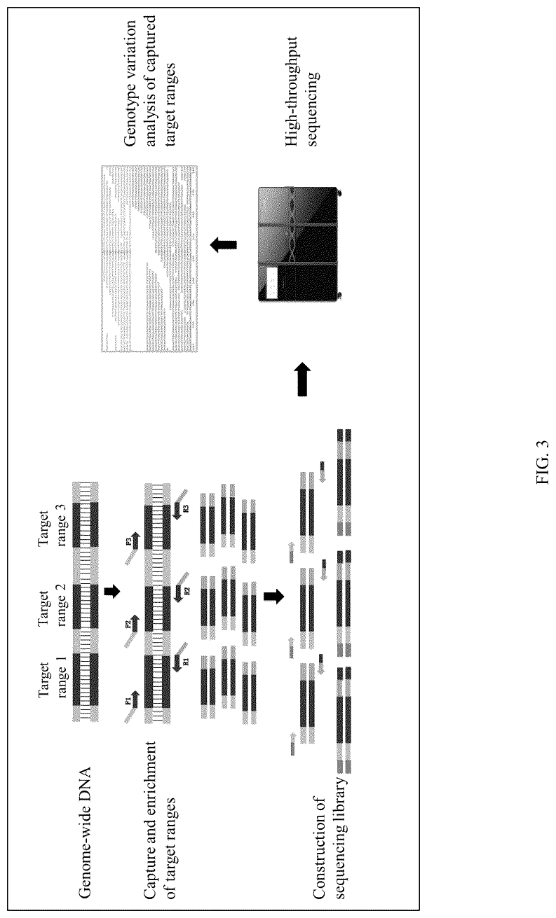

77 samples of Brassica napus L. are detected, and the detection method is specifically shown in FIG. 3. The detection method includes: DNA extraction, target segment amplification, ligating sequencing adapters and amplification, library quality inspection and sequencing analysis.

### 1. DNA Extraction

The DNA extraction is performed on the 77 samples of Brassica napus L. by using a method independently developed by Huazhi Biotechnology Co., Ltd to obtain DNA samples. An enzyme-linked immunosorbent assay (ELISA) instrument is used for quality control to obtain qualified DNA samples.

### 2. Target Segment Amplification

(1) PCR amplification is performed on the qualified DNA samples by using a 1.5 K liquid-phase gene chip of Brassica napus L. independently developed by Huazhi Biotechnology Co., Ltd to obtain amplification products.
(2) Segment size quality control is performed on the amplification products to obtain target segment products with 300 bp.

### 3. Ligating Sequencing Adapters and Amplification

The obtained target segment products are ligated with sequencing adapters, and a second round of PCR amplification is performed on the target segment products ligated with the sequencing adapters to obtain a final library.

### 4. Library Quality Inspection

A concentration of DNA in the library is measured by using QubitFluorometric Quantitation (Thermo Fisher Scientific Inc.), and agarose gel electrophoresis is performed on the DNA segments in the library to detect whether sizes of the DNA segments in the library is between 300 to 400 bp.

### 5. DNA Hybridization Capture and Library Sequencing

The constructed DNA library is sequenced by a sequencing group of Huazhi Biotechnology Co., Ltd to obtain sequencing data.

### 6. Genotypic Data Analysis

After quality control of sequencing data by using a fast quality control (FastQC) method, the sequencing data is aligned to the reference genome by using default parameters of burrows-wheeler aligner (BWA), and SNP identification is performed on the aligned sequencing data by using a genome analysis tool kit (GATK) software, so as to perform cluster analysis on the 77 samples.

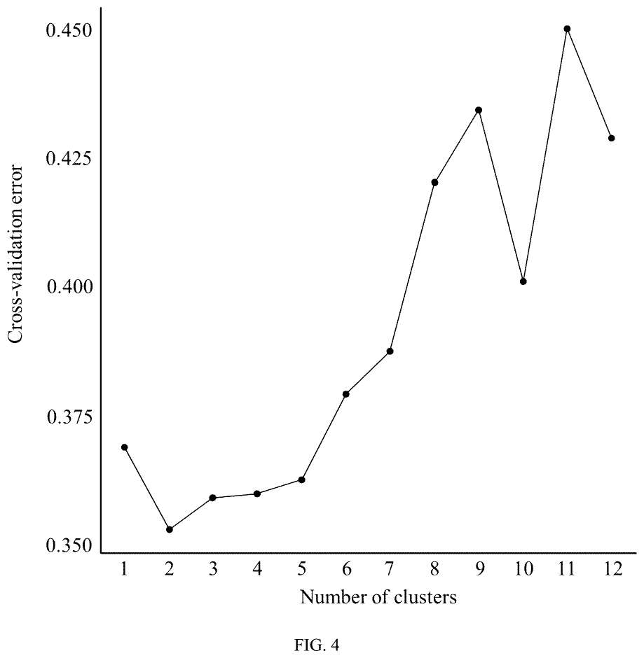

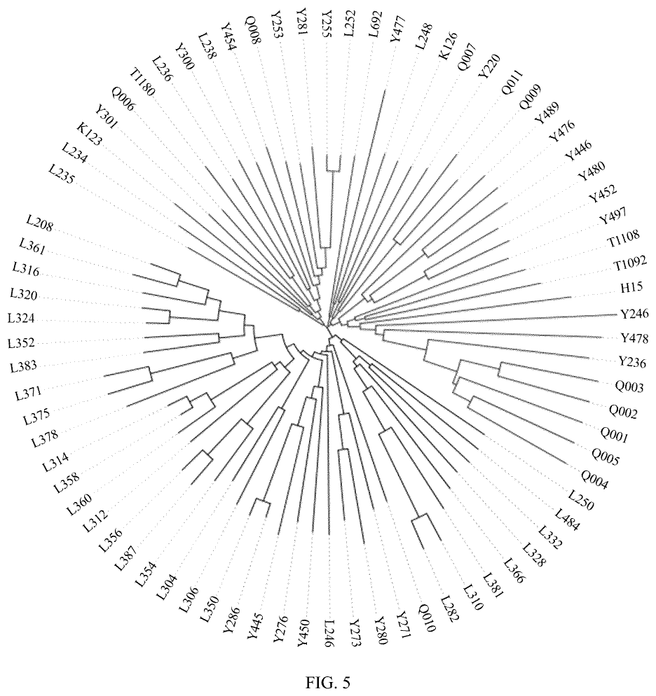

The cluster analysis results are shown in FIG. 4 and FIG. 5. It can be seen from FIG. 4 that, when the number of clusters is 2, the cross-validation error is the lowest (i.e., the K value, also referred to as the number of clusters, is optimal), and a classification effect of materials is optimal. The materials are divided into two types. It can be seen from FIG. 5 that corresponding samples in the two types of materials have significant genetic background difference, and the difference increases with the increase of classification trees. According to the above results, material strains with great genetic difference can be selected as core parents for subsequent breeding selection.

The results indicate that the SNP molecular marker combination of Brassica napus L. of the disclosure can be effectively used in cluster analysis and paternity testing of Brassica napus L.

### Embodiment 3

An application method of the SNP molecular marker combination in paternity testing of Brassica napus L. is provided, and the method includes the following steps.

2 Brassica napus L. materials with known paternity are provided, and the 2 materials include: a receptor parent and a multi-generation recurrent strain.

In step (1), the 2 materials are sequenced (the sequencing method is the same as that in the embodiment 2) to obtain sequencing data, and the sequencing data is analyzed by using a 1.5 K chip to obtain locus alignment results.

In step (2), statistical analysis is performed on the locus alignment results to obtain a response rate of the locus alignment results as 97.41%.

In step (3), chromosome mapping analysis is performed on the loci, to thereby visually analyze positions of differential loci in the chromosomes of the 2 materials.

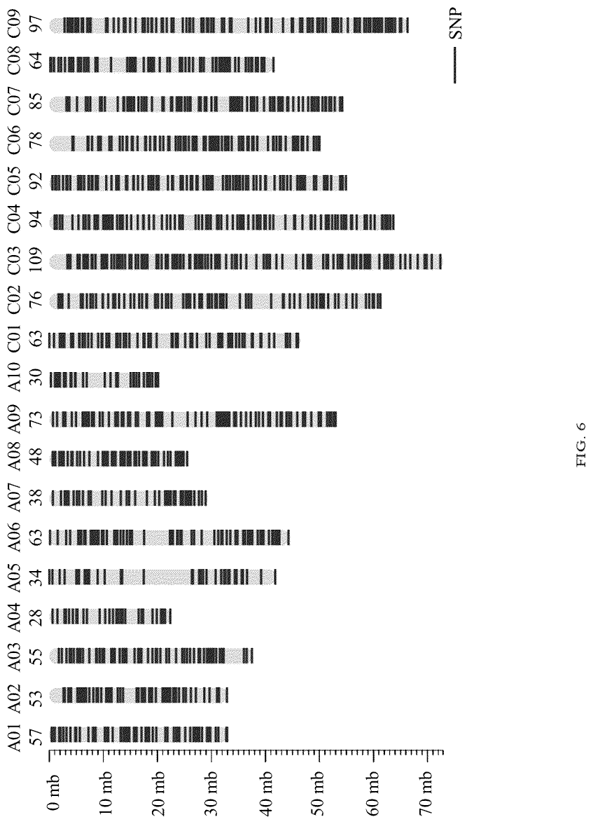

A result of the visually analyze positions of differential loci in the chromosomes of the 2 materials is shown in FIG. 6. It can be seen from FIG. 6 that: A, the similarity between the 2 materials is very high, with only a small number of loci showing differences; B, there are significant segment differences between the 2 materials on C03 and C09, with few others; and C, the reply chromosome segments can be considered for marker detection, so as to efficiently achieve improvement of the target segments.

The results indicate that the SNP molecular marker combination of the disclosure can be effectively used in paternity testing of Brassica napus L.

The above detailed description of the embodiments of the disclosure is provided in conjunction with the drawings, but the disclosure is not limited to the aforementioned embodiments. Within the scope of knowledge possessed by those skilled in the art, various changes can be made without departing from the purpose of the disclosure. In addition, without conflict, the embodiments of the disclosure and the features in the embodiments can be combined with each other.

What is claimed is:

1. A single nucleotide polymorphism (SNP) molecular marker combination of Brassica napus L., wherein the SNP molecular marker combination comprises 1407 SNP molecular markers, and the 1407 SNP molecular markers are shown in BNC001-1407; physical positions of the 1407 SNP molecular markers are determined by sequence alignment based on a reference genome Brana_Dar_ViG of the Brassica napus L., and specific locus information of the 1407 SNP molecular markers is as follows:

| Maeker | SNP | | Physical |
|---|---|---|---|
| name | locus | Chromosome | position |
| BN0001 | [A/G] | A01 | 129037 |
| BN0002 | [G/T] | A01 | 428925 |
| BN0003 | [A/T] | A01 | 613260 |
| BN0004 | [G/C] | A01 | 766699 |
| BN0005 | [G/A] | A01 | 1025737 |
| BN0006 | [C/T] | A01 | 1437661 |
| BN0007 | [A/G] | A01 | 1836189 |
| BN0008 | [G/C] | A01 | 2101529 |
| BN0009 | [T/C] | A01 | 2621155 |
| BN0010 | [C/T] | A01 | 3189305 |
| BN0011 | [A/G] | A01 | 3484531 |
| BN0012 | [C/A] | A01 | 3915906 |
| BN0013 | [A/G] | A01 | 4798105 |
| BN0014 | [A/G] | A01 | 4995490 |
| BN0015 | [G/C] | A01 | 5656374 |
| BN0016 | [T/C] | A01 | 6256801 |
| BN0017 | [G/T] | A01 | 6554695 |
| BN0018 | [C/G] | A01 | 6975952 |
| BN0019 | [A/G] | A01 | 7256244 |
| BN0020 | [G/A] | A01 | 8082266 |
| BN0021 | [T/A] | A01 | 8422189 |
| BN0022 | [T/A] | A01 | 8580582 |
| BN0023 | [C/G] | A01 | 9496906 |
| BN0024 | [G/A] | A01 | 9964538 |
| BN0025 | [T/G] | A01 | 10278462 |
| BN0026 | [T/C] | A01 | 10691388 |
| BN0027 | [C/T] | A01 | 10914494 |
| BN0028 | [C/A] | A01 | 11735978 |
| BN0029 | [C/T] | A01 | 12075653 |
| BN0030 | [C/T] | A01 | 13215910 |
| BN0031 | [T/A] | A01 | 13369626 |
| BN0032 | [T/A] | A01 | 13792030 |
| BN0033 | [C/G] | A01 | 14076059 |
| BN0034 | [T/A] | A01 | 14242025 |
| BN0035 | [A/G] | A01 | 14531087 |
| BN0036 | [C/T] | A01 | 14591462 |
| BN0037 | [G/T] | A01 | 14820924 |
| BN0038 | [G/C] | A01 | 15092908 |
| BN0039 | [T/C] | A01 | 15384809 |
| BN0040 | [G/T] | A01 | 15676588 |
| BN0041 | [T/A] | A01 | 15908466 |
| BN0042 | [A/G] | A01 | 16165301 |
| BN0043 | [C/T] | A01 | 17080618 |
| BN0044 | [G/A] | A01 | 17831711 |
| BN0045 | [A/C] | A01 | 17938462 |
| BN0046 | [T/G] | A01 | 18314187 |
| BN0047 | [C/T] | A01 | 18477965 |
| BN0048 | [T/C] | A01 | 19157860 |
| BN0049 | [A/G] | A01 | 19811843 |
| BN0050 | [C/T] | A01 | 20761418 |
| BN0051 | [T/C] | A01 | 21872829 |
| BN0052 | [T/C] | A01 | 22498537 |
| BN0053 | [G/A] | A01 | 23055560 |
| BN0054 | [T/C] | A01 | 23481834 |
| BN0055 | [G/C] | A01 | 23789453 |
| BN0056 | [T/A] | A01 | 24071025 |
| BN0057 | [G/A] | A01 | 25164508 |
| BN0058 | [G/A] | A01 | 25312032 |
| BN0059 | [T/A] | A01 | 26099200 |
| BN0060 | [T/C] | A01 | 26704687 |
| BN0061 | [C/T] | A01 | 27108445 |
| BN0062 | [T/C] | A01 | 27500324 |
| BN0063 | [T/G] | A01 | 27754935 |
| BN0064 | [G/T] | A01 | 28259675 |
| BN0065 | [G/T] | A01 | 28940588 |
| BN0066 | [C/T] | A01 | 29186931 |
| BN0067 | [T/G] | A01 | 29586142 |
| BN0068 | [T/A] | A01 | 29799486 |
| BN0069 | [T/A] | A01 | 30473575 |
| BN0070 | [A/C] | A01 | 30822101 |
| BN0071 | [G/C] | A01 | 30991803 |
| BN0072 | [C/T] | A01 | 31286361 |
| BN0073 | [G/T] | A01 | 32753867 |
| BN0074 | [C/G] | A01 | 32920903 |
| BN0075 | [T/A] | A02 | 1477438 |
| BN0076 | [A/G] | A02 | 1706353 |
| BN0077 | [T/C] | A02 | 2202267 |
| BN0078 | [A/T] | A02 | 2647003 |
| BN0079 | [G/T] | A02 | 2880803 |
| BN0080 | [G/A] | A02 | 3337050 |
| BN0081 | [C/T] | A02 | 3537064 |
| BN0082 | [T/C] | A02 | 3742268 |
| BN0083 | [A/C] | A02 | 4037886 |
| BN0084 | [G/C] | A02 | 5199998 |
| BN0085 | [T/C] | A02 | 5422396 |
| BN0086 | [T/C] | A02 | 5771631 |
| BN0087 | [G/A] | A02 | 5994344 |
| BN0088 | [C/T] | A02 | 6247196 |
| BN0089 | [G/C] | A02 | 6536986 |
| BN0090 | [A/C] | A02 | 6818209 |
| BN0091 | [G/C] | A02 | 7142397 |
| BN0092 | [A/G] | A02 | 7416706 |
| BN0093 | [T/A] | A02 | 7939578 |
| BN0094 | [T/C] | A02 | 8157741 |
| BN0095 | [T/G] | A02 | 8755245 |
| BN0096 | [C/T] | A02 | 9028967 |
| BN0097 | [T/C] | A02 | 9629117 |
| BN0098 | [G/A] | A02 | 10535585 |
| BN0099 | [G/A] | A02 | 10911720 |
| BN0100 | [A/C] | A02 | 11254116 |
| BN0101 | [A/G] | A02 | 11526796 |
| BN0102 | [T/C] | A02 | 11950079 |
| BN0103 | [T/C] | A02 | 12672987 |
| BN0104 | [T/C] | A02 | 13136845 |
| BN0105 | [C/T] | A02 | 13651745 |
| BN0106 | [G/T] | A02 | 14111450 |
| BN0107 | [C/T] | A02 | 14715499 |
| BN0108 | [A/T] | A02 | 16173502 |
| BN0109 | [A/G] | A02 | 16525868 |
| BN0110 | [T/C] | A02 | 17111749 |
| BN0111 | [G/T] | A02 | 17460550 |
| BN0112 | [A/G] | A02 | 17715705 |
| BN0113 | [T/A] | A02 | 18015759 |
| BN0114 | [T/A] | A02 | 18243304 |
| BN0115 | [A/C] | A02 | 18489557 |
| BN0116 | [G/A] | A02 | 18811701 |
| BN0117 | [T/C] | A02 | 19028233 |
| BN0118 | [A/C] | A02 | 19496673 |
| BN0119 | [G/T] | A02 | 19729156 |
| BN0120 | [A/G] | A02 | 20966584 |
| BN0121 | [T/G] | A02 | 21300405 |
| BN0122 | [G/A] | A02 | 21502165 |
| BN0123 | [C/T] | A02 | 21776475 |
| BN0124 | [G/A] | A02 | 21992906 |
| BN0125 | [G/T] | A02 | 22188583 |
| BN0126 | [A/G] | A02 | 22381160 |
| BN0127 | [G/A] | A02 | 22940568 |
| BN0128 | [C/T] | A02 | 23457378 |
| BN0129 | [A/G] | A02 | 24005725 |
| BN0130 | [T/C] | A02 | 26214476 |
| BN0131 | [T/C] | A02 | 27131996 |
| BN0132 | [G/A] | A02 | 27732157 |
| BN0133 | [C/A] | A02 | 28695870 |
| BN0134 | [C/A] | A02 | 29661805 |
| BN0135 | [C/T] | A02 | 29930522 |
| BN0136 | [C/T] | A02 | 31050927 |
| BN0137 | [C/A] | A02 | 31409538 |
| BN0138 | [G/C] | A02 | 32880693 |
| BN0139 | [A/G] | A03 | 406149 |
| BN0140 | [A/T] | A03 | 614038 |
| BN0141 | [C/T] | A03 | 1770044 |
| BN0142 | [A/C] | A03 | 2321281 |
| BN0143 | [C/T] | A03 | 2655754 |
| BN0144 | [G/C] | A03 | 2866250 |
| BN0145 | [A/G] | A03 | 3186370 |
| BN0146 | [A/T] | A03 | 3778663 |
| BN0147 | [T/C] | A03 | 4062184 |
| BN0148 | [C/A] | A03 | 4299121 |
| BN0149 | [A/T] | A03 | 4528203 |
| BN0150 | [A/C] | A03 | 5174561 |
| BN0151 | [T/G] | A03 | 5484353 |
| BN0152 | [C/T] | A03 | 5752271 |
| BN0153 | [T/C] | A03 | 6715834 |
| BN0154 | [C/T] | A03 | 7407617 |
| BN0155 | [G/A] | A03 | 8844122 |
| BN0156 | [A/G] | A03 | 9326938 |
| BN0157 | [G/A] | A03 | 9801889 |
| BN0158 | [A/G] | A03 | 10152463 |
| BN0159 | [T/C] | A03 | 11478790 |
| BN0160 | [G/A] | A03 | 11727707 |
| BN0161 | [T/C] | A03 | 12109548 |
| BN0162 | [G/T] | A03 | 13024780 |
| BN0163 | [G/A] | A03 | 13567802 |
| BN0164 | [A/G] | A03 | 13959325 |
| BN0165 | [T/C] | A03 | 14275400 |
| BN0166 | [G/A] | A03 | 15788344 |
| BN0167 | [A/G] | A03 | 16465433 |
| BN0168 | [T/C] | A03 | 16728477 |
| BN0169 | [G/C] | A03 | 17059091 |
| BN0170 | [A/C] | A03 | 18279238 |
| BN0171 | [A/G] | A03 | 19095438 |
| BN0172 | [C/G] | A03 | 19716647 |
| BN0173 | [A/T] | A03 | 19959329 |
| BN0174 | [T/C] | A03 | 20562498 |
| BN0175 | [T/C] | A03 | 20997431 |
| BN0176 | [C/G] | A03 | 21668732 |
| BN0177 | [C/T] | A03 | 22160666 |
| BN0178 | [C/T] | A03 | 23469478 |
| BN0179 | [A/G] | A03 | 23854578 |
| BN0180 | [A/T] | A03 | 24268573 |
| BN0181 | [T/C] | A03 | 24618632 |
| BN0182 | [A/G] | A03 | 25024759 |
| BN0183 | [A/G] | A03 | 25502419 |
| BN0184 | [T/G] | A03 | 26062918 |
| BN0185 | [A/G] | A03 | 26781654 |
| BN0186 | [T/G] | A03 | 27289278 |
| BN0187 | [G/C] | A03 | 27644223 |
| BN0188 | [T/C] | A03 | 27920290 |
| BN0189 | [G/A] | A03 | 28624068 |
| BN0190 | [C/T] | A03 | 29181558 |
| BN0191 | [G/A] | A03 | 29521804 |
| BN0192 | [G/A] | A03 | 29732498 |
| BN0193 | [A/G] | A03 | 30075229 |
| BN0194 | [G/T] | A03 | 30399550 |
| BN0195 | [A/G] | A03 | 30882866 |
| BN0196 | [T/C] | A03 | 31559217 |
| BN0197 | [C/G] | A03 | 31977200 |
| BN0198 | [T/G] | A03 | 32271145 |
| BN0199 | [G/A] | A03 | 33680884 |
| BN0200 | [T/C] | A03 | 35229675 |
| BN0201 | [C/T] | A03 | 36037542 |
| BN0202 | [G/C] | A03 | 36544390 |
| BN0203 | [A/T] | A03 | 37502943 |
| BN0204 | [G/C] | A04 | 605905 |
| BN0205 | [G/T] | A04 | 1516508 |
| BN0206 | [A/T] | A04 | 2817214 |
| BN0207 | [C/T] | A04 | 3084717 |
| BN0208 | [C/T] | A04 | 3665795 |
| BN0209 | [A/G] | A04 | 4365699 |
| BN0210 | [T/C] | A04 | 5021135 |
| BN0211 | [C/A] | A04 | 5210952 |
| BN0212 | [A/G] | A04 | 6339746 |
| BN0213 | [T/G] | A04 | 6975258 |
| BN0214 | [T/C] | A04 | 7783900 |
| BN0215 | [A/G] | A04 | 9325873 |
| BN0216 | [C/A] | A04 | 10357480 |
| BN0217 | [G/T] | A04 | 11134891 |
| BN0218 | [C/T] | A04 | 11793239 |
| BN0219 | [G/A] | A04 | 12433336 |
| BN0220 | [G/A] | A04 | 12752347 |
| BN0221 | [A/G] | A04 | 13213440 |
| BN0222 | [T/C] | A04 | 13585869 |
| BN0223 | [A/G] | A04 | 14061987 |
| BN0224 | [C/T] | A04 | 14699418 |
| BN0225 | [C/T] | A04 | 15810172 |
| BN0226 | [G/A] | A04 | 16551694 |
| BN0227 | [G/T] | A04 | 17086259 |
| BN0228 | [A/G] | A04 | 17366895 |
| BN0229 | [C/G] | A04 | 18673865 |
| BN0230 | [G/A] | A04 | 19109792 |
| BN0231 | [A/G] | A04 | 19438862 |
| BN0232 | [C/T] | A04 | 19516841 |
| BN0233 | [A/G] | A04 | 19894101 |
| BN0234 | [G/A] | A04 | 20296015 |
| BN0235 | [C/A] | A04 | 20678658 |
| BN0236 | [C/T] | A04 | 21001498 |
| BN0237 | [C/T] | A04 | 21427386 |
| BN0238 | [G/A] | A04 | 22221101 |
| BN0239 | [C/T] | A04 | 22397348 |
| BN0240 | [A/G] | A05 | 10899 |
| BN0241 | [A/G] | A05 | 390148 |
| BN0242 | [G/T] | A05 | 582316 |
| BN0243 | [A/G] | A05 | 1086270 |
| BN0244 | [A/G] | A05 | 1811198 |
| BN0245 | [C/T] | A05 | 1924532 |
| BN0246 | [G/A] | A05 | 2661901 |
| BN0247 | [A/T] | A05 | 2841541 |
| BN0248 | [C/T] | A05 | 4323400 |
| BN0249 | [T/C] | A05 | 5009580 |
| BN0250 | [C/G] | A05 | 5499857 |
| BN0251 | [A/G] | A05 | 6977523 |
| BN0252 | [T/C] | A05 | 7372610 |
| BN0253 | [T/C] | A05 | 8749499 |
| BN0254 | [C/A] | A05 | 8959263 |
| BN0255 | [G/A] | A05 | 10258530 |
| BN0256 | [T/G] | A05 | 11564316 |
| BN0257 | [C/G] | A05 | 11883288 |
| BN0258 | [A/G] | A05 | 12452356 |
| BN0259 | [A/C] | A05 | 13061358 |
| BN0260 | [G/C] | A05 | 13258563 |
| BN0261 | [A/T] | A05 | 13491544 |
| BN0262 | [G/A] | A05 | 13553669 |
| BN0263 | [G/T] | A05 | 15101609 |
| BN0264 | [A/G] | A05 | 16208236 |
| BN0265 | [C/T] | A05 | 17277143 |
| BN0266 | [C/A] | A05 | 17491278 |
| BN0267 | [G/C] | A05 | 19577916 |
| BN0268 | [G/A] | A05 | 19958998 |
| BN0269 | [C/T] | A05 | 20998579 |
| BN0270 | [C/T] | A05 | 25415343 |
| BN0271 | [A/C] | A05 | 26386555 |
| BN0272 | [G/A] | A05 | 26590152 |
| BN0273 | [C/T] | A05 | 27379626 |
| BN0274 | [A/T] | A05 | 27731387 |
| BN0275 | [C/T] | A05 | 28069804 |
| BN0276 | [G/A] | A05 | 28481737 |
| BN0277 | [C/T] | A05 | 29107210 |
| BN0278 | [C/T] | A05 | 30788919 |
| BN0279 | [C/T] | A05 | 31816944 |
| BN0280 | [A/G] | A05 | 32603435 |
| BN0281 | [G/A] | A05 | 33110405 |
| BN0282 | [C/G] | A05 | 33357533 |
| BN0283 | [A/G] | A05 | 34099497 |
| BN0284 | [A/C] | A05 | 34341577 |
| BN0285 | [T/C] | A05 | 34663148 |
| BN0286 | [A/C] | A05 | 35608329 |
| BN0287 | [C/T] | A05 | 35829658 |
| BN0288 | [G/A] | A05 | 36312828 |
| BN0289 | [C/A] | A05 | 36600227 |
| BN0290 | [A/T] | A05 | 37611553 |
| BN0291 | [T/G] | A05 | 38108829 |
| BN0292 | [A/G] | A05 | 38865463 |
| BN0293 | [C/A] | A05 | 39209611 |
| BN0294 | [A/T] | A05 | 40839055 |
| BN0295 | [G/A] | A05 | 41820634 |
| BN0296 | [C/A] | A06 | 129694 |
| BN0297 | [A/G] | A06 | 1567300 |
| BN0298 | [A/G] | A06 | 3130615 |
| BN0299 | [G/T] | A06 | 3605047 |
| BN0300 | [T/A] | A06 | 3859392 |
| BN0301 | [A/G] | A06 | 5116532 |
| BN0302 | [G/A] | A06 | 5366079 |
| BN0303 | [C/T] | A06 | 5642020 |
| BN0304 | [C/A] | A06 | 5926997 |
| BN0305 | [A/G] | A06 | 6472206 |
| BN0306 | [G/A] | A06 | 6710627 |
| BN0307 | [G/A] | A06 | 7680229 |
| BN0308 | [T/A] | A06 | 7877587 |
| BN0309 | [C/T] | A06 | 8506048 |
| BN0310 | [T/G] | A06 | 8893548 |
| BN0311 | [A/G] | A06 | 9127109 |
| BN0312 | [T/C] | A06 | 9759098 |
| BN0313 | [A/G] | A06 | 10041482 |
| BN0314 | [A/C] | A06 | 10381729 |
| BN0315 | [G/A] | A06 | 10659873 |
| BN0316 | [C/A] | A06 | 11032570 |
| BN0317 | [T/G] | A06 | 12034384 |
| BN0318 | [A/G] | A06 | 12272687 |
| BN0319 | [A/C] | A06 | 12613541 |
| BN0320 | [C/A] | A06 | 13203632 |
| BN0321 | [C/A] | A06 | 13734879 |
| BN0322 | [T/C] | A06 | 14356865 |
| BN0323 | [G/A] | A06 | 14874993 |
| BN0324 | [A/G] | A06 | 15045436 |
| BN0325 | [G/A] | A06 | 15305898 |
| BN0326 | [G/T] | A06 | 17550566 |
| BN0327 | [A/G] | A06 | 22600936 |
| BN0328 | [G/T] | A06 | 22883532 |
| BN0329 | [T/C] | A06 | 23682059 |
| BN0330 | [A/G] | A06 | 23737247 |
| BN0331 | [C/G] | A06 | 24238616 |
| BN0332 | [G/T] | A06 | 25517201 |
| BN0333 | [C/A] | A06 | 26347762 |
| BN0334 | [G/C] | A06 | 26648095 |
| BN0335 | [G/A] | A06 | 26802195 |
| BN0336 | [T/C] | A06 | 28207078 |
| BN0337 | [C/G] | A06 | 31270202 |
| BN0338 | [G/A] | A06 | 31570643 |
| BN0339 | [T/G] | A06 | 31833475 |
| BN0340 | [G/A] | A06 | 32052205 |
| BN0341 | [G/A] | A06 | 32843815 |
| BN0342 | [C/T] | A06 | 33508966 |
| BN0343 | [C/T] | A06 | 34300539 |
| BN0344 | [G/A] | A06 | 34469363 |
| BN0345 | [G/T] | A06 | 34832781 |
| BN0346 | [G/T] | A06 | 35268689 |
| BN0347 | [A/C] | A06 | 35895088 |
| BN0348 | [G/A] | A06 | 36240436 |
| BN0349 | [A/G] | A06 | 36750388 |
| BN0350 | [A/G] | A06 | 36978489 |
| BN0351 | [G/A] | A06 | 37325744 |
| BN0352 | [T/C] | A06 | 37579242 |
| BN0353 | [G/A] | A06 | 38576970 |
| BN0354 | [T/C] | A06 | 39088036 |
| BN0355 | [A/G] | A06 | 39303110 |
| BN0356 | [T/C] | A06 | 39645025 |
| BN0357 | [C/G] | A06 | 40659271 |
| BN0358 | [G/T] | A06 | 41278454 |
| BN0359 | [G/A] | A06 | 41495428 |
| BN0360 | [T/G] | A06 | 41948893 |
| BN0361 | [G/A] | A06 | 42246097 |
| BN0362 | [T/C] | A06 | 42596937 |
| BN0363 | [T/C] | A06 | 44269738 |
| BN0364 | [C/A] | A07 | 668085 |
| BN0365 | [A/G] | A07 | 2171771 |
| BN0366 | [G/C] | A07 | 2862401 |
| BN0367 | [G/C] | A07 | 2965262 |
| BN0368 | [T/C] | A07 | 3217388 |
| BN0369 | [A/G] | A07 | 3565365 |
| BN0370 | [A/G] | A07 | 3738488 |
| BN0371 | [A/T] | A07 | 4040624 |
| BN0372 | [G/A] | A07 | 4514906 |
| BN0373 | [C/G] | A07 | 5125677 |
| BN0374 | [A/T] | A07 | 5550115 |
| BN0375 | [T/G] | A07 | 6234141 |
| BN0376 | [A/C] | A07 | 7290359 |
| BN0377 | [C/T] | A07 | 7692692 |
| BN0378 | [T/C] | A07 | 7937632 |
| BN0379 | [T/A] | A07 | 8276541 |
| BN0380 | [G/A] | A07 | 9044074 |
| BN0381 | [C/T] | A07 | 9815556 |
| BN0382 | [C/G] | A07 | 10425619 |
| BN0383 | [T/C] | A07 | 11527525 |
| BN0384 | [G/T] | A07 | 11978225 |
| BN0385 | [A/T] | A07 | 12115653 |
| BN0386 | [T/A] | A07 | 12854015 |
| BN0387 | [C/T] | A07 | 13237954 |
| BN0388 | [G/A] | A07 | 13893669 |
| BN0389 | [C/T] | A07 | 14324741 |
| BN0390 | [C/T] | A07 | 14784694 |
| BN0391 | [G/A] | A07 | 15226791 |
| BN0392 | [A/T] | A07 | 15898732 |
| BN0393 | [C/A] | A07 | 17352868 |
| BN0394 | [A/G] | A07 | 18072617 |
| BN0395 | [C/A] | A07 | 19546201 |
| BN0396 | [G/A] | A07 | 20348366 |
| BN0397 | [T/C] | A07 | 20718232 |
| BN0398 | [G/A] | A07 | 21442948 |
| BN0399 | [T/G] | A07 | 21729267 |
| BN0400 | [T/C] | A07 | 22066928 |
| BN0401 | [T/C] | A07 | 22482144 |
| BN0402 | [C/A] | A07 | 23044434 |
| BN0403 | [C/T] | A07 | 23347169 |
| BN0404 | [C/T] | A07 | 23969864 |
| BN0405 | [G/A] | A07 | 24340883 |
| BN0406 | [C/T] | A07 | 24707123 |
| BN0407 | [G/T] | A07 | 25187263 |
| BN0408 | [C/G] | A07 | 25478920 |
| BN0409 | [G/A] | A07 | 25894147 |
| BN0410 | [C/A] | A07 | 26262783 |
| BN0411 | [A/G] | A07 | 26589215 |
| BN0412 | [G/A] | A07 | 26845711 |
| BN0413 | [T/C] | A07 | 27000458 |
| BN0414 | [A/T] | A07 | 27698572 |
| BN0415 | [A/G] | A07 | 28244204 |
| BN0416 | [A/G] | A07 | 28937837 |
| BN0417 | [A/T] | A07 | 29275848 |
| BN0418 | [C/T] | A08 | 631906 |
| BN0419 | [A/G] | A08 | 779607 |
| BN0420 | [T/A] | A08 | 1123921 |
| BN0421 | [A/G] | A08 | 1856707 |
| BN0422 | [C/T] | A08 | 2233185 |
| BN0423 | [G/A] | A08 | 2485603 |
| BN0424 | [G/A] | A08 | 2666843 |
| BN0425 | [T/A] | A08 | 3414886 |
| BN0426 | [A/G] | A08 | 4098983 |
| BN0427 | [C/T] | A08 | 4312251 |
| BN0428 | [C/T] | A08 | 5696369 |
| BN0429 | [G/A] | A08 | 6440305 |
| BN0430 | [C/T] | A08 | 7424918 |
| BN0431 | [G/A] | A08 | 7569419 |
| BN0432 | [G/A] | A08 | 8046074 |
| BN0433 | [C/A] | A08 | 9018021 |
| BN0434 | [G/C] | A08 | 9907323 |
| BN0435 | [A/G] | A08 | 10220612 |
| BN0436 | [C/A] | A08 | 10582809 |
| BN0437 | [T/C] | A08 | 11589004 |
| BN0438 | [T/G] | A08 | 11899471 |
| BN0439 | [A/G] | A08 | 12279983 |
| BN0440 | [C/G] | A08 | 13205651 |
| BN0441 | [C/G] | A08 | 13430201 |
| BN0442 | [T/A] | A08 | 13718301 |
| BN0443 | [G/A] | A08 | 13949898 |
| BN0444 | [G/A] | A08 | 14181039 |
| BN0445 | [T/C] | A08 | 14496188 |
| BN0446 | [T/G] | A08 | 14723327 |
| BN0447 | [T/C] | A08 | 15030289 |
| BN0448 | [G/A] | A08 | 15778329 |
| BN0449 | [G/C] | A08 | 16072164 |
| BN0450 | [T/C] | A08 | 16616518 |
| BN0451 | [G/A] | A08 | 17376251 |
| BN0452 | [A/T] | A08 | 17698239 |
| BN0453 | [T/A] | A08 | 18007170 |
| BN0454 | [G/A] | A08 | 18963249 |
| BN0455 | [G/A] | A08 | 19251338 |
| BN0456 | [G/C] | A08 | 19670443 |
| BN0457 | [G/A] | A08 | 20006619 |
| BN0458 | [A/G] | A08 | 20166166 |
| BN0459 | [C/A] | A08 | 20962898 |
| BN0460 | [T/C] | A08 | 21241059 |
| BN0461 | [G/A] | A08 | 21955248 |
| BN0462 | [C/T] | A08 | 22433691 |
| BN0463 | [G/C] | A08 | 23418525 |
| BN0464 | [C/T] | A08 | 23772405 |
| BN0465 | [C/T] | A08 | 24056759 |
| BN0466 | [A/G] | A08 | 24439272 |
| BN0467 | [C/G] | A08 | 24855357 |
| BN0468 | [C/T] | A08 | 25528663 |
| BN0469 | [A/T] | A09 | 683941 |
| BN0470 | [A/G] | A09 | 1444300 |
| BN0471 | [T/C] | A09 | 2168403 |
| BN0472 | [T/C] | A09 | 2575381 |
| BN0473 | [G/T] | A09 | 2991392 |
| BN0474 | [G/A] | A09 | 3356668 |
| BN0475 | [T/A] | A09 | 4172077 |
| BN0476 | [C/T] | A09 | 4945897 |
| BN0477 | [G/A] | A09 | 6184467 |
| BN0478 | [T/C] | A09 | 6479356 |
| BN0479 | [T/C] | A09 | 6860948 |
| BN0480 | [T/A] | A09 | 7148472 |
| BN0481 | [C/T] | A09 | 7345802 |
| BN0482 | [G/T] | A09 | 7901623 |
| BN0483 | [C/T] | A09 | 8256377 |
| BN0484 | [T/G] | A09 | 8902282 |
| BN0485 | [C/T] | A09 | 9351158 |
| BN0486 | [G/A] | A09 | 9891239 |
| BN0487 | [A/G] | A09 | 11033545 |
| BN0488 | [A/C] | A09 | 11299500 |
| BN0489 | [A/G] | A09 | 11558775 |
| BN0490 | [A/G] | A09 | 12223445 |
| BN0491 | [T/C] | A09 | 12407616 |
| BN0492 | [T/C] | A09 | 12708854 |
| BN0493 | [C/A] | A09 | 13404932 |
| BN0494 | [T/A] | A09 | 13740653 |
| BN0495 | [A/G] | A09 | 14589940 |
| BN0496 | [C/A] | A09 | 16414756 |
| BN0497 | [T/C] | A09 | 17591969 |
| BN0498 | [G/A] | A09 | 18102674 |
| BN0499 | [A/T] | A09 | 18503422 |
| BN0500 | [C/T] | A09 | 19728271 |
| BN0501 | [C/T] | A09 | 20030529 |
| BN0502 | [G/C] | A09 | 20374382 |
| BN0503 | [G/A] | A09 | 20620568 |
| BN0504 | [A/G] | A09 | 20910738 |
| BN0505 | [C/T] | A09 | 21142497 |
| BN0506 | [G/A] | A09 | 22780701 |
| BN0507 | [G/A] | A09 | 25604964 |
| BN0508 | [C/T] | A09 | 27068826 |
| BN0509 | [A/T] | A09 | 28187150 |
| BN0510 | [T/C] | A09 | 29227077 |
| BN0511 | [A/T] | A09 | 30357361 |
| BN0512 | [T/A] | A09 | 30995903 |
| BN0513 | [A/C] | A09 | 31291528 |
| BN0514 | [C/T] | A09 | 31496175 |
| BN0515 | [A/G] | A09 | 31852632 |
| BN0516 | [A/G] | A09 | 32180423 |
| BN0517 | [C/G] | A09 | 32521467 |
| BN0518 | [T/G] | A09 | 32701933 |
| BN0519 | [A/C] | A09 | 32973381 |
| BN0520 | [C/T] | A09 | 33322885 |
| BN0521 | [G/A] | A09 | 34222096 |
| BN0522 | [C/A] | A09 | 34563605 |
| BN0523 | [T/C] | A09 | 35114800 |
| BN0524 | [C/T] | A09 | 35473201 |
| BN0525 | [C/A] | A09 | 36422923 |
| BN0526 | [C/T] | A09 | 37285873 |
| BN0527 | [C/A] | A09 | 38228054 |
| BN0528 | [G/A] | A09 | 38803185 |
| BN0529 | [C/T] | A09 | 39545280 |
| BN0530 | [C/T] | A09 | 40546328 |
| BN0531 | [A/C] | A09 | 40863518 |
| BN0532 | [T/A] | A09 | 42121681 |
| BN0533 | [G/A] | A09 | 43046674 |
| BN0534 | [A/C] | A09 | 43345325 |
| BN0535 | [G/A] | A09 | 43899902 |
| BN0536 | [T/A] | A09 | 44372059 |
| BN0537 | [C/A] | A09 | 44760067 |
| BN0538 | [G/C] | A09 | 45896819 |
| BN0539 | [G/A] | A09 | 46510941 |
| BN0540 | [C/A] | A09 | 47352882 |
| BN0541 | [C/A] | A09 | 47982192 |
| BN0542 | [C/T] | A09 | 48365495 |
| BN0543 | [A/T] | A09 | 48495780 |
| BN0544 | [C/T] | A09 | 48909687 |
| BN0545 | [C/T] | A09 | 49559700 |
| BN0546 | [G/T] | A09 | 49860591 |
| BN0547 | [G/A] | A09 | 50270440 |
| BN0548 | [G/C] | A09 | 51140947 |
| BN0549 | [A/G] | A09 | 51420252 |
| BN0550 | [T/G] | A09 | 51844532 |
| BN0551 | [C/T] | A09 | 52210525 |
| BN0552 | [G/C] | A09 | 52674285 |
| BN0553 | [T/C] | A09 | 53001318 |
| BN0554 | [T/G] | A10 | 293245 |
| BN0555 | [A/G] | A10 | 519105 |
| BN0556 | [G/T] | A10 | 1058320 |
| BN0557 | [C/G] | A10 | 1391954 |
| BN0558 | [G/T] | A10 | 1644622 |
| BN0559 | [A/G] | A10 | 1967264 |
| BN0560 | [A/C] | A10 | 2719788 |
| BN0561 | [A/G] | A10 | 3047568 |
| BN0562 | [G/T] | A10 | 3952229 |
| BN0563 | [T/G] | A10 | 4772203 |
| BN0564 | [T/C] | A10 | 4869005 |
| BN0565 | [G/A] | A10 | 6255054 |
| BN0566 | [C/T] | A10 | 6929260 |
| BN0567 | [C/T] | A10 | 7261047 |
| BN0568 | [A/C] | A10 | 9246270 |
| BN0569 | [A/G] | A10 | 10276067 |
| BN0570 | [A/G] | A10 | 10496200 |
| BN0571 | [C/T] | A10 | 11338128 |
| BN0572 | [G/C] | A10 | 11657997 |
| BN0573 | [G/A] | A10 | 12393777 |
| BN0574 | [T/G] | A10 | 12699587 |
| BN0575 | [A/G] | A10 | 13590381 |
| BN0576 | [T/C] | A10 | 13867538 |
| BN0577 | [A/T] | A10 | 14549628 |
| BN0578 | [C/T] | A10 | 15067397 |
| BN0579 | [C/G] | A10 | 15570898 |
| BN0580 | [G/A] | A10 | 15664032 |
| BN0581 | [A/G] | A10 | 16149149 |
| BN0582 | [T/A] | A10 | 16624529 |
| BN0583 | [A/T] | A10 | 17510683 |
| BN0584 | [C/A] | A10 | 18111756 |
| BN0585 | [T/A] | A10 | 18413345 |
| BN0586 | [G/A] | A10 | 18649419 |
| BN0587 | [G/A] | A10 | 19080114 |
| BN0588 | [G/T] | A10 | 19695808 |
| BN0589 | [A/G] | A10 | 19942920 |
| BN0590 | [T/A] | A10 | 20146695 |
| BN0591 | [G/C] | C01 | 8708 |
| BN0592 | [A/G] | C01 | 871836 |
| BN0593 | [G/A] | C01 | 1955455 |
| BN0594 | [G/A] | C01 | 2327745 |
| BN0595 | [T/C] | C01 | 2792164 |
| BN0596 | [C/T] | C01 | 3495216 |
| BN0597 | [T/C] | C01 | 4045803 |
| BN0598 | [T/C] | C01 | 4412983 |
| BN0599 | [C/T] | C01 | 4594315 |
| BN0600 | [A/C] | C01 | 5545134 |
| BN0601 | [C/T] | C01 | 5619624 |
| BN0602 | [A/G] | C01 | 6163796 |
| BN0603 | [G/A] | C01 | 6604131 |
| BN0604 | [C/T] | C01 | 7206428 |
| BN0605 | [G/A] | C01 | 7835279 |
| BN0606 | [C/A] | C01 | 9051193 |
| BN0607 | [G/T] | C01 | 9678225 |
| BN0608 | [T/A] | C01 | 9815380 |
| BN0609 | [T/A] | C01 | 10211368 |
| BN0610 | [A/G] | C01 | 10591847 |
| BN0611 | [T/A] | C01 | 10988051 |
| BN0612 | [A/C] | C01 | 11098406 |
| BN0613 | [C/A] | C01 | 12420842 |
| BN0614 | [C/A] | C01 | 12691362 |
| BN0615 | [G/A] | C01 | 13143732 |
| BN0616 | [G/C] | C01 | 13798685 |
| BN0617 | [G/A] | C01 | 14236494 |
| BN0618 | [C/T] | C01 | 14830021 |
| BN0619 | [G/T] | C01 | 16334917 |
| BN0620 | [C/T] | C01 | 17448440 |
| BN0621 | [T/G] | C01 | 17630184 |
| BN0622 | [C/T] | C01 | 18350592 |
| BN0623 | [T/C] | C01 | 19874920 |
| BN0624 | [A/C] | C01 | 20685434 |
| BN0625 | [G/C] | C01 | 22656715 |
| BN0626 | [T/A] | C01 | 23418558 |
| BN0627 | [A/G] | C01 | 23779523 |
| BN0628 | [A/C] | C01 | 25160006 |
| BN0629 | [G/C] | C01 | 26133860 |
| BN0630 | [A/G] | C01 | 26477005 |
| BN0631 | [T/G] | C01 | 27342159 |
| BN0632 | [A/G] | C01 | 29054098 |
| BN0633 | [A/G] | C01 | 29436093 |
| BN0634 | [C/T] | C01 | 29663679 |
| BN0635 | [A/C] | C01 | 30916636 |
| BN0636 | [C/T] | C01 | 31290776 |
| BN0637 | [A/C] | C01 | 31587341 |
| BN0638 | [T/A] | C01 | 32012992 |
| BN0639 | [T/G] | C01 | 33144223 |
| BN0640 | [C/T] | C01 | 33457842 |
| BN0641 | [A/G] | C01 | 33847873 |
| BN0642 | [A/G] | C01 | 34577660 |
| BN0643 | [G/A] | C01 | 35155759 |
| BN0644 | [T/G] | C01 | 35747007 |
| BN0645 | [C/A] | C01 | 36155019 |
| BN0646 | [T/G] | C01 | 37492587 |
| BN0647 | [C/A] | C01 | 37675108 |
| BN0648 | [C/T] | C01 | 38729557 |
| BN0649 | [C/G] | C01 | 39093335 |
| BN0650 | [T/A] | C01 | 39248157 |
| BN0651 | [T/G] | C01 | 39700878 |
| BN0652 | [G/C] | C01 | 40131785 |
| BN0653 | [A/T] | C01 | 40693595 |
| BN0654 | [T/C] | C01 | 40993969 |
| BN0655 | [T/C] | C01 | 41631061 |
| BN0656 | [C/A] | C01 | 42667054 |
| BN0657 | [C/T] | C01 | 43422067 |
| BN0658 | [G/A] | C01 | 43789928 |
| BN0659 | [G/T] | C01 | 44078953 |
| BN0660 | [C/A] | C01 | 44544380 |
| BN0661 | [A/T] | C01 | 45774141 |
| BN0662 | [C/T] | C01 | 46031495 |
| BN0663 | [C/A] | C01 | 46425279 |
| BN0664 | [C/T] | C02 | 446631 |
| BN0665 | [G/C] | C02 | 1433716 |
| BN0666 | [A/C] | C02 | 2060222 |
| BN0667 | [C/T] | C02 | 3596587 |
| BN0668 | [T/C] | C02 | 4259143 |
| BN0669 | [G/A] | C02 | 4512184 |
| BN0670 | [C/T] | C02 | 5848345 |
| BN0671 | [G/A] | C02 | 6211025 |
| BN0672 | [C/T] | C02 | 6957317 |
| BN0673 | [G/C] | C02 | 7624307 |
| BN0674 | [T/C] | C02 | 8071605 |
| BN0675 | [T/A] | C02 | 8657730 |
| BN0676 | [A/G] | C02 | 9881890 |
| BN0677 | [A/T] | C02 | 9923641 |
| BN0678 | [G/T] | C02 | 10916858 |
| BN0679 | [T/A] | C02 | 11841573 |
| BN0680 | [C/A] | C02 | 12212092 |
| BN0681 | [C/T] | C02 | 12920024 |
| BN0682 | [A/T] | C02 | 13367776 |
| BN0683 | [A/T] | C02 | 13880427 |
| BN0684 | [C/T] | C02 | 15053881 |
| BN0685 | [T/C] | C02 | 15721033 |
| BN0686 | [A/T] | C02 | 16250462 |
| BN0687 | [T/C] | C02 | 16643147 |
| BN0688 | [A/T] | C02 | 17185103 |
| BN0689 | [A/G] | C02 | 17335325 |
| BN0690 | [A/G] | C02 | 17962007 |
| BN0691 | [A/C] | C02 | 18098011 |
| BN0692 | [A/G] | C02 | 18448522 |
| BN0693 | [G/T] | C02 | 19699835 |
| BN0694 | [C/A] | C02 | 20122470 |
| BN0695 | [A/G] | C02 | 20845138 |
| BN0696 | [C/G] | C02 | 21605022 |
| BN0697 | [T/C] | C02 | 22020996 |
| BN0698 | [C/T] | C02 | 22606544 |
| BN0699 | [C/A] | C02 | 23891184 |
| BN0700 | [G/A] | C02 | 24787280 |
| BN0701 | [G/A] | C02 | 25343290 |
| BN0702 | [A/T] | C02 | 26083694 |
| BN0703 | [G/T] | C02 | 26868853 |
| BN0704 | [G/T] | C02 | 27791884 |
| BN0705 | [T/A] | C02 | 28011953 |
| BN0706 | [G/T] | C02 | 29339848 |
| BN0707 | [C/T] | C02 | 29648935 |
| BN0708 | [G/C] | C02 | 30508042 |
| BN0709 | [A/G] | C02 | 30715190 |
| BN0710 | [C/T] | C02 | 31020826 |
| BN0711 | [T/C] | C02 | 31619084 |
| BN0712 | [T/G] | C02 | 31904664 |
| BN0713 | [C/A] | C02 | 32248097 |
| BN0714 | [A/G] | C02 | 32792129 |
| BN0715 | [A/T] | C02 | 34119723 |
| BN0716 | [A/G] | C02 | 35276154 |
| BN0717 | [G/C] | C02 | 35598006 |
| BN0718 | [T/C] | C02 | 36417665 |
| BN0719 | [G/A] | C02 | 36884482 |
| BN0720 | [G/A] | C02 | 37368822 |
| BN0721 | [A/G] | C02 | 41069401 |
| BN0722 | [T/C] | C02 | 43238326 |
| BN0723 | [T/G] | C02 | 43410556 |
| BN0724 | [T/C] | C02 | 44514126 |
| BN0725 | [C/T] | C02 | 45350655 |
| BN0726 | [C/T] | C02 | 46392239 |
| BN0727 | [A/C] | C02 | 47919432 |
| BN0728 | [A/C] | C02 | 48597389 |
| BN0729 | [T/G] | C02 | 48852375 |
| BN0730 | [C/T] | C02 | 49082420 |
| BN0731 | [C/T] | C02 | 50134514 |
| BN0732 | [G/A] | C02 | 50485510 |
| BN0733 | [G/A] | C02 | 50803847 |
| BN0734 | [G/A] | C02 | 51625416 |
| BN0735 | [G/A] | C02 | 52894189 |
| BN0736 | [C/A] | C02 | 53525738 |
| BN0737 | [G/C] | C02 | 54975581 |
| BN0738 | [G/A] | C02 | 56138778 |
| BN0739 | [C/A] | C02 | 56845571 |
| BN0740 | [T/A] | C02 | 59077729 |
| BN0741 | [C/T] | C02 | 59209808 |
| BN0742 | [C/A] | C02 | 59861437 |
| BN0743 | [T/A] | C02 | 60085790 |
| BN0744 | [A/G] | C02 | 60820081 |
| BN0745 | [G/T] | C02 | 61293668 |
| BN0746 | [T/C] | C03 | 283272 |
| BN0747 | [T/C] | C03 | 793411 |
| BN0748 | [T/C] | C03 | 1191506 |
| BN0749 | [C/T] | C03 | 1893029 |
| BN0750 | [T/G] | C03 | 3356771 |
| BN0751 | [A/T] | C03 | 3722727 |
| BN0752 | [G/A] | C03 | 4186013 |
| BN0753 | [A/G] | C03 | 4448485 |
| BN0754 | [A/G] | C03 | 5233815 |
| BN0755 | [C/T] | C03 | 5635209 |
| BN0756 | [T/C] | C03 | 6329226 |
| BN0757 | [T/C] | C03 | 6444305 |
| BN0758 | [G/T] | C03 | 6774161 |
| BN0759 | [T/A] | C03 | 7023943 |
| BN0760 | [G/A] | C03 | 7143884 |
| BN0761 | [C/A] | C03 | 7836413 |
| BN0762 | [T/C] | C03 | 8039469 |
| BN0763 | [G/A] | C03 | 8562357 |
| BN0764 | [T/C] | C03 | 9045009 |
| BN0765 | [C/A] | C03 | 9703152 |
| BN0766 | [T/C] | C03 | 9824172 |
| BN0767 | [G/T] | C03 | 10129035 |
| BN0768 | [T/C] | C03 | 10456184 |
| BN0769 | [T/C] | C03 | 10700776 |
| BN0770 | [C/T] | C03 | 11520947 |
| BN0771 | [C/G] | C03 | 12034638 |
| BN0772 | [T/C] | C03 | 12552333 |
| BN0773 | [T/A] | C03 | 12839422 |
| BN0774 | [T/A] | C03 | 13118824 |
| BN0775 | [C/T] | C03 | 13530789 |
| BN0776 | [A/T] | C03 | 14031619 |
| BN0777 | [T/G] | C03 | 14705816 |
| BN0778 | [G/C] | C03 | 14847352 |
| BN0779 | [T/C] | C03 | 15203527 |
| BN0780 | [C/T] | C03 | 15994977 |
| BN0781 | [T/A] | C03 | 16450327 |
| BN0782 | [T/C] | C03 | 17083944 |
| BN0783 | [C/G] | C03 | 17369619 |
| BN0784 | [A/T] | C03 | 18032825 |
| BN0785 | [T/C] | C03 | 18175861 |
| BN0786 | [A/G] | C03 | 19844731 |
| BN0787 | [T/C] | C03 | 20218854 |
| BN0788 | [T/G] | C03 | 20794891 |
| BN0789 | [T/G] | C03 | 21553624 |
| BN0790 | [T/C] | C03 | 21944345 |
| BN0791 | [T/C] | C03 | 22811433 |
| BN0792 | [G/T] | C03 | 23174218 |
| BN0793 | [T/A] | C03 | 23359002 |
| BN0794 | [T/G] | C03 | 23687057 |
| BN0795 | [T/C] | C03 | 24134015 |
| BN0796 | [G/A] | C03 | 24343380 |
| BN0797 | [C/T] | C03 | 24850311 |
| BN0798 | [A/G] | C03 | 25926846 |
| BN0799 | [A/T] | C03 | 26092564 |
| BN0800 | [G/T] | C03 | 26723128 |
| BN0801 | [G/T] | C03 | 26966616 |
| BN0802 | [A/T] | C03 | 27278142 |
| BN0803 | [T/C] | C03 | 27715670 |
| BN0804 | [G/A] | C03 | 28006864 |
| BN0805 | [T/C] | C03 | 28330380 |
| BN0806 | [G/C] | C03 | 28864233 |
| BN0807 | [G/A] | C03 | 29358422 |
| BN0808 | [A/G] | C03 | 29980301 |
| BN0809 | [A/G] | C03 | 30716901 |
| BN0810 | [A/T] | C03 | 31057779 |
| BN0811 | [G/A] | C03 | 31222222 |
| BN0812 | [A/G] | C03 | 31541772 |
| BN0813 | [A/T] | C03 | 32730913 |
| BN0814 | [C/T] | C03 | 33797411 |
| BN0815 | [C/T] | C03 | 34348931 |
| BN0816 | [G/T] | C03 | 34716788 |
| BN0817 | [C/T] | C03 | 35240370 |
| BN0818 | [T/G] | C03 | 35435358 |
| BN0819 | [T/G] | C03 | 36462990 |
| BN0820 | [C/A] | C03 | 37305168 |
| BN0821 | [A/G] | C03 | 38328252 |
| BN0822 | [A/T] | C03 | 39685447 |
| BN0823 | [A/C] | C03 | 40174316 |
| BN0824 | [T/C] | C03 | 40436236 |
| BN0825 | [T/C] | C03 | 40832750 |
| BN0826 | [G/A] | C03 | 41174437 |
| BN0827 | [T/C] | C03 | 42037503 |
| BN0828 | [G/C] | C03 | 43143358 |
| BN0829 | [T/G] | C03 | 45682356 |
| BN0830 | [C/T] | C03 | 47803683 |
| BN0831 | [G/A] | C03 | 48176867 |
| BN0832 | [G/A] | C03 | 48469865 |
| BN0833 | [C/T] | C03 | 51281833 |
| BN0834 | [G/A] | C03 | 52595712 |
| BN0835 | [T/C] | C03 | 53389165 |
| BN0836 | [A/T] | C03 | 53847216 |
| BN0837 | [C/T] | C03 | 54244309 |
| BN0838 | [A/C] | C03 | 55381982 |
| BN0839 | [G/T] | C03 | 56045422 |
| BN0840 | [G/A] | C03 | 56387955 |
| BN0841 | [C/A] | C03 | 56865699 |
| BN0842 | [C/A] | C03 | 57546575 |
| BN0843 | [C/A] | C03 | 57741931 |
| BN0844 | [T/A] | C03 | 58349173 |
| BN0845 | [G/A] | C03 | 58604707 |
| BN0846 | [A/G] | C03 | 58750265 |
| BN0847 | [G/A] | C03 | 59077018 |
| BN0848 | [G/A] | C03 | 59355736 |
| BN0849 | [A/G] | C03 | 59623072 |
| BN0850 | [G/C] | C03 | 61088262 |
| BN0851 | [C/A] | C03 | 61373006 |
| BN0852 | [T/G] | C03 | 62013450 |
| BN0853 | [G/T] | C03 | 62383376 |
| BN0854 | [T/C] | C03 | 62705127 |
| BN0855 | [A/G] | C03 | 63221795 |
| BN0856 | [A/T] | C03 | 63590742 |
| BN0857 | [C/T] | C03 | 64089999 |
| BN0858 | [T/C] | C03 | 64990674 |
| BN0859 | [G/A] | C03 | 65877705 |
| BN0860 | [G/C] | C03 | 66817363 |
| BN0861 | [G/T] | C03 | 68192503 |
| BN0862 | [A/C] | C03 | 69462861 |
| BN0863 | [C/A] | C03 | 69840304 |
| BN0864 | [T/A] | C03 | 70158981 |
| BN0865 | [G/T] | C03 | 70578297 |
| BN0866 | [C/G] | C03 | 70782661 |
| BN0867 | [C/T] | C03 | 71054951 |
| BN0868 | [A/G] | C03 | 72375920 |
| BN0869 | [T/A] | C03 | 72867662 |
| BN0870 | [G/C] | C04 | 995760 |
| BN0871 | [C/T] | C04 | 1400602 |
| BN0872 | [A/G] | C04 | 1960851 |
| BN0873 | [A/G] | C04 | 2424972 |
| BN0874 | [G/A] | C04 | 4294617 |
| BN0875 | [T/C] | C04 | 5406538 |
| BN0876 | [T/C] | C04 | 6334157 |
| BN0877 | [G/A] | C04 | 6735716 |
| BN0878 | [C/T] | C04 | 7278952 |
| BN0879 | [T/C] | C04 | 7401501 |
| BN0880 | [A/G] | C04 | 8382077 |
| BN0881 | [A/G] | C04 | 8732463 |
| BN0882 | [T/A] | C04 | 9006183 |
| BN0883 | [T/C] | C04 | 9300205 |
| BN0884 | [T/C] | C04 | 9995799 |
| BN0885 | [G/C] | C04 | 10479058 |
| BN0886 | [A/C] | C04 | 10725573 |
| BN0887 | [A/G] | C04 | 11022285 |
| BN0888 | [G/A] | C04 | 11138648 |
| BN0889 | [C/T] | C04 | 12568524 |
| BN0890 | [G/T] | C04 | 13079880 |
| BN0891 | [C/G] | C04 | 13442727 |
| BN0892 | [G/T] | C04 | 13611098 |
| BN0893 | [G/T] | C04 | 14355281 |
| BN0894 | [T/C] | C04 | 15157327 |
| BN0895 | [T/G] | C04 | 16372862 |
| BN0896 | [A/G] | C04 | 17381862 |
| BN0897 | [T/G] | C04 | 17486079 |
| BN0898 | [A/G] | C04 | 18461994 |
| BN0899 | [A/G] | C04 | 19353982 |
| BN0900 | [C/T] | C04 | 19942430 |
| BN0901 | [T/C] | C04 | 20847446 |
| BN0902 | [A/G] | C04 | 21771065 |
| BN0903 | [G/T] | C04 | 22415089 |
| BN0904 | [T/A] | C04 | 23238753 |
| BN0905 | [G/C] | C04 | 24704796 |
| BN0906 | [G/A] | C04 | 27090952 |
| BN0907 | [G/T] | C04 | 27241498 |
| BN0908 | [A/G] | C04 | 27659190 |
| BN0909 | [C/G] | C04 | 28299122 |
| BN0910 | [C/T] | C04 | 29044180 |
| BN0911 | [C/A] | C04 | 29428417 |
| BN0912 | [T/C] | C04 | 29886413 |
| BN0913 | [C/A] | C04 | 31049038 |
| BN0914 | [G/T] | C04 | 31361167 |
| BN0915 | [C/T] | C04 | 31832393 |
| BN0916 | [C/G] | C04 | 32795738 |
| BN0917 | [T/G] | C04 | 32921459 |
| BN0918 | [A/G] | C04 | 33970759 |
| BN0919 | [A/G] | C04 | 34826469 |
| BN0920 | [A/C] | C04 | 35268016 |
| BN0921 | [T/C] | C04 | 35610048 |
| BN0922 | [G/A] | C04 | 35939884 |
| BN0923 | [T/C] | C04 | 36841052 |
| BN0924 | [G/C] | C04 | 37769811 |
| BN0925 | [G/A] | C04 | 38423726 |
| BN0926 | [G/A] | C04 | 38690510 |
| BN0927 | [G/A] | C04 | 39084985 |
| BN0928 | [T/C] | C04 | 39815017 |
| BN0929 | [G/T] | C04 | 40077317 |
| BN0930 | [C/T] | C04 | 40822742 |
| BN0931 | [C/T] | C04 | 41085145 |
| BN0932 | [A/T] | C04 | 41461884 |
| BN0933 | [G/A] | C04 | 41923499 |
| BN0934 | [G/C] | C04 | 42775603 |
| BN0935 | [C/A] | C04 | 43700308 |
| BN0936 | [A/G] | C04 | 44429632 |
| BN0937 | [T/A] | C04 | 45321143 |
| BN0938 | [A/G] | C04 | 45497566 |
| BN0939 | [A/T] | C04 | 46856810 |
| BN0940 | [G/A] | C04 | 48361577 |
| BN0941 | [A/C] | C04 | 48539855 |
| BN0942 | [T/C] | C04 | 48979973 |
| BN0943 | [G/A] | C04 | 49192842 |
| BN0944 | [T/C] | C04 | 50289946 |
| BN0945 | [A/C] | C04 | 51189585 |
| BN0946 | [G/A] | C04 | 51452289 |
| BN0947 | [A/T] | C04 | 51729695 |
| BN0948 | [T/C] | C04 | 52354747 |
| BN0949 | [A/G] | C04 | 52930373 |
| BN0950 | [G/A] | C04 | 53393153 |
| BN0951 | [T/C] | C04 | 54056106 |
| BN0952 | [C/T] | C04 | 54503099 |
| BN0953 | [G/A] | C04 | 55856030 |
| BN0954 | [T/C] | C04 | 56042753 |
| BN0955 | [A/T] | C04 | 56459789 |
| BN0956 | [G/T] | C04 | 56898406 |
| BN0957 | [T/G] | C04 | 58368239 |
| BN0958 | [C/A] | C04 | 58779466 |
| BN0959 | [G/A] | C04 | 59482111 |
| BN0960 | [A/G] | C04 | 60214734 |
| BN0961 | [C/T] | C04 | 60753969 |
| BN0962 | [G/C] | C04 | 61594114 |
| BN0963 | [G/T] | C04 | 61702147 |
| BN0964 | [A/G] | C04 | 62157069 |
| BN0965 | [A/G] | C04 | 62363676 |
| BN0966 | [G/T] | C04 | 62679888 |
| BN0967 | [T/C] | C04 | 63139920 |
| BN0968 | [G/A] | C04 | 63687680 |
| BN0969 | [C/T] | C05 | 565559 |
| BN0970 | [C/A] | C05 | 1075034 |
| BN0971 | [C/T] | C05 | 1333060 |
| BN0972 | [C/A] | C05 | 1702479 |
| BN0973 | [G/T] | C05 | 1823004 |
| BN0974 | [T/G] | C05 | 2549850 |
| BN0975 | [G/C] | C05 | 3167714 |
| BN0976 | [C/A] | C05 | 3376130 |
| BN0977 | [G/C] | C05 | 4027941 |
| BN0978 | [G/A] | C05 | 5340419 |
| BN0979 | [C/T] | C05 | 5428513 |
| BN0980 | [C/T] | C05 | 5763702 |
| BN0981 | [A/G] | C05 | 5949947 |
| BN0982 | [T/C] | C05 | 6465242 |
| BN0983 | [C/T] | C05 | 7266432 |
| BN0984 | [C/T] | C05 | 7363682 |
| BN0985 | [G/A] | C05 | 7864864 |
| BN0986 | [T/C] | C05 | 7999522 |
| BN0987 | [C/G] | C05 | 8593851 |
| BN0988 | [T/C] | C05 | 8704056 |
| BN0989 | [C/T] | C05 | 8983821 |
| BN0990 | [T/A] | C05 | 10554193 |
| BN0991 | [A/C] | C05 | 11519749 |
| BN0992 | [A/T] | C05 | 12407458 |
| BN0993 | [C/A] | C05 | 12715647 |
| BN0994 | [C/T] | C05 | 12856641 |
| BN0995 | [C/A] | C05 | 14204463 |
| BN0996 | [C/T] | C05 | 14436369 |
| BN0997 | [A/G] | C05 | 15555371 |
| BN0998 | [C/A] | C05 | 15647726 |
| BN0999 | [A/G] | C05 | 16415555 |
| BN1000 | [G/T] | C05 | 17280802 |
| BN1001 | [A/T] | C05 | 18378861 |
| BN1002 | [G/A] | C05 | 18799222 |
| BN1003 | [A/G] | C05 | 18928599 |
| BN1004 | [A/G] | C05 | 19305763 |
| BN1005 | [A/G] | C05 | 19844481 |
| BN1006 | [A/T] | C05 | 20006835 |
| BN1007 | [C/T] | C05 | 20233529 |
| BN1008 | [G/C] | C05 | 21964136 |
| BN1009 | [C/T] | C05 | 22739956 |
| BN1010 | [C/T] | C05 | 22900999 |
| BN1011 | [C/T] | C05 | 23262361 |
| BN1012 | [A/G] | C05 | 24308126 |
| BN1013 | [G/A] | C05 | 25454587 |
| BN1014 | [C/A] | C05 | 26182201 |
| BN1015 | [C/G] | C05 | 26467598 |
| BN1016 | [C/G] | C05 | 27248026 |
| BN1017 | [C/T] | C05 | 27454601 |
| BN1018 | [G/A] | C05 | 28549353 |
| BN1019 | [A/T] | C05 | 29074087 |
| BN1020 | [A/G] | C05 | 29228647 |
| BN1021 | [G/A] | C05 | 29561576 |
| BN1022 | [A/C] | C05 | 29932394 |
| BN1023 | [A/G] | C05 | 32145906 |
| BN1024 | [G/A] | C05 | 32727296 |
| BN1025 | [A/C] | C05 | 32985417 |
| BN1026 | [G/T] | C05 | 33453957 |
| BN1027 | [A/G] | C05 | 34035803 |
| BN1028 | [C/T] | C05 | 34507047 |
| BN1029 | [T/A] | C05 | 34699231 |
| BN1030 | [T/C] | C05 | 35100756 |
| BN1031 | [A/T] | C05 | 35316632 |
| BN1032 | [C/G] | C05 | 35839965 |
| BN1033 | [T/G] | C05 | 36414172 |
| BN1034 | [A/G] | C05 | 36808715 |
| BN1035 | [C/T] | C05 | 37808693 |
| BN1036 | [C/T] | C05 | 38346379 |
| BN1037 | [G/A] | C05 | 39074064 |
| BN1038 | [C/T] | C05 | 39954408 |
| BN1039 | [A/G] | C05 | 41699687 |
| BN1040 | [T/C] | C05 | 42739412 |
| BN1041 | [G/T] | C05 | 43232743 |
| BN1042 | [C/A] | C05 | 43425261 |
| BN1043 | [T/C] | C05 | 44034668 |
| BN1044 | [T/A] | C05 | 44664713 |
| BN1045 | [C/A] | C05 | 45027392 |
| BN1046 | [C/T] | C05 | 45901215 |
| BN1047 | [C/G] | C05 | 46352592 |
| BN1048 | [T/A] | C05 | 46708275 |
| BN1049 | [C/A] | C05 | 46941796 |
| BN1050 | [C/G] | C05 | 47312938 |
| BN1051 | [G/A] | C05 | 49395415 |
| BN1052 | [A/T] | C05 | 50856718 |
| BN1053 | [T/A] | C05 | 51096431 |
| BN1054 | [A/G] | C05 | 51492585 |
| BN1055 | [C/G] | C05 | 52173378 |
| BN1056 | [C/T] | C05 | 54006722 |
| BN1057 | [C/A] | C05 | 54311819 |
| BN1058 | [A/G] | C05 | 54906161 |
| BN1059 | [G/A] | C06 | 4295963 |
| BN1060 | [A/T] | C06 | 4495052 |
| BN1061 | [G/A] | C06 | 5451146 |
| BN1062 | [A/T] | C06 | 6262483 |
| BN1063 | [A/C] | C06 | 6650602 |
| BN1064 | [G/A] | C06 | 7093445 |
| BN1065 | [C/A] | C06 | 7269980 |
| BN1066 | [G/T] | C06 | 7922352 |
| BN1067 | [C/A] | C06 | 8388494 |
| BN1068 | [G/T] | C06 | 8627166 |
| BN1069 | [G/T] | C06 | 9058908 |
| BN1070 | [A/G] | C06 | 9223161 |
| BN1071 | [G/A] | C06 | 9536029 |
| BN1072 | [T/C] | C06 | 10650709 |
| BN1073 | [C/A] | C06 | 11131970 |
| BN1074 | [C/T] | C06 | 11517831 |
| BN1075 | [G/T] | C06 | 11623464 |
| BN1076 | [A/G] | C06 | 12321749 |
| BN1077 | [G/T] | C06 | 13034901 |
| BN1078 | [G/A] | C06 | 13574003 |
| BN1079 | [T/C] | C06 | 14305562 |
| BN1080 | [C/T] | C06 | 14415859 |
| BN1081 | [A/C] | C06 | 15277464 |
| BN1082 | [G/A] | C06 | 15456411 |
| BN1083 | [T/G] | C06 | 15703128 |
| BN1084 | [C/T] | C06 | 15948506 |
| BN1085 | [G/A] | C06 | 16335997 |
| BN1086 | [C/G] | C06 | 17750558 |
| BN1087 | [C/T] | C06 | 17899659 |
| BN1088 | [C/T] | C06 | 18661395 |
| BN1089 | [A/T] | C06 | 19635730 |
| BN1090 | [T/G] | C06 | 20034183 |
| BN1091 | [C/T] | C06 | 20502253 |
| BN1092 | [C/T] | C06 | 22029339 |
| BN1093 | [A/G] | C06 | 22916161 |
| BN1094 | [T/C] | C06 | 23399985 |
| BN1095 | [A/T] | C06 | 23873225 |
| BN1096 | [A/G] | C06 | 24073443 |
| BN1097 | [T/A] | C06 | 24333233 |
| BN1098 | [C/T] | C06 | 24871829 |
| BN1099 | [C/A] | C06 | 25258827 |
| BN1100 | [A/G] | C06 | 25832607 |
| BN1101 | [C/T] | C06 | 26544808 |
| BN1102 | [G/A] | C06 | 26886927 |
| BN1103 | [C/T] | C06 | 27073498 |
| BN1104 | [T/C] | C06 | 27252149 |
| BN1105 | [C/G] | C06 | 28582234 |
| BN1106 | [C/T] | C06 | 29152897 |
| BN1107 | [C/G] | C06 | 29696118 |
| BN1108 | [G/A] | C06 | 29888627 |
| BN1109 | [G/T] | C06 | 30178506 |
| BN1110 | [A/G] | C06 | 30760973 |
| BN1111 | [G/A] | C06 | 31149536 |
| BN1112 | [T/G] | C06 | 31344860 |
| BN1113 | [C/T] | C06 | 31943521 |
| BN1114 | [T/C] | C06 | 32237886 |
| BN1115 | [A/C] | C06 | 32526730 |
| BN1116 | [G/A] | C06 | 32837641 |
| BN1117 | [T/A] | C06 | 33042448 |
| BN1118 | [T/C] | C06 | 33170630 |
| BN1119 | [G/C] | C06 | 33763816 |
| BN1120 | [G/C] | C06 | 35104213 |
| BN1121 | [G/C] | C06 | 35538572 |
| BN1122 | [A/C] | C06 | 35895789 |
| BN1123 | [T/C] | C06 | 36670077 |
| BN1124 | [A/G] | C06 | 36912105 |
| BN1125 | [G/A] | C06 | 37581336 |
| BN1126 | [A/G] | C06 | 37810453 |
| BN1127 | [T/G] | C06 | 38465249 |
| BN1128 | [G/A] | C06 | 38511501 |
| BN1129 | [C/T] | C06 | 40176880 |
| BN1130 | [A/G] | C06 | 40480463 |
| BN1131 | [T/C] | C06 | 41422906 |
| BN1132 | [T/C] | C06 | 41534728 |
| BN1133 | [C/T] | C06 | 42057537 |
| BN1134 | [T/G] | C06 | 42606103 |
| BN1135 | [T/A] | C06 | 42857680 |
| BN1136 | [A/T] | C06 | 45113731 |
| BN1137 | [T/C] | C06 | 45404469 |
| BN1138 | [G/A] | C06 | 45730301 |
| BN1139 | [A/T] | C06 | 46704746 |
| BN1140 | [G/T] | C06 | 46983810 |
| BN1141 | [T/G] | C06 | 47793183 |
| BN1142 | [T/A] | C06 | 48421426 |
| BN1143 | [T/A] | C06 | 48958810 |
| BN1144 | [T/A] | C06 | 49307537 |
| BN1145 | [A/G] | C06 | 49418342 |
| BN1146 | [A/C] | C06 | 49821766 |
| BN1147 | [G/A] | C06 | 50004479 |
| BN1148 | [T/A] | C07 | 3185168 |
| BN1149 | [T/C] | C07 | 3462520 |
| BN1150 | [C/A] | C07 | 3719282 |
| BN1151 | [C/T] | C07 | 5137118 |
| BN1152 | [C/T] | C07 | 6774856 |
| BN1153 | [T/A] | C07 | 7150922 |
| BN1154 | [C/T] | C07 | 7594885 |
| BN1155 | [G/C] | C07 | 7897356 |
| BN1156 | [T/G] | C07 | 9401404 |
| BN1157 | [T/C] | C07 | 9506508 |
| BN1158 | [G/A] | C07 | 9729125 |
| BN1159 | [T/G] | C07 | 10294157 |
| BN1160 | [A/G] | C07 | 11242970 |
| BN1161 | [T/C] | C07 | 12672736 |
| BN1162 | [A/T] | C07 | 13729250 |
| BN1163 | [A/C] | C07 | 13857620 |
| BN1164 | [A/C] | C07 | 14460382 |
| BN1165 | [A/G] | C07 | 14603257 |
| BN1166 | [T/C] | C07 | 14923357 |
| BN1167 | [G/A] | C07 | 15260645 |
| BN1168 | [T/C] | C07 | 15683443 |
| BN1169 | [T/C] | C07 | 16508903 |
| BN1170 | [C/G] | C07 | 17091508 |
| BN1171 | [A/G] | C07 | 17530607 |
| BN1172 | [G/A] | C07 | 17985851 |
| BN1173 | [G/A] | C07 | 20940241 |
| BN1174 | [A/G] | C07 | 21185852 |
| BN1175 | [C/G] | C07 | 22530834 |
| BN1176 | [G/C] | C07 | 22757098 |
| BN1177 | [T/A] | C07 | 23602300 |
| BN1178 | [A/G] | C07 | 23767891 |
| BN1179 | [T/C] | C07 | 24113860 |
| BN1180 | [G/T] | C07 | 24364883 |
| BN1181 | [G/A] | C07 | 24472900 |
| BN1182 | [A/G] | C07 | 25085563 |
| BN1183 | [C/T] | C07 | 25580594 |
| BN1184 | [C/T] | C07 | 25995417 |
| BN1185 | [G/A] | C07 | 26147121 |
| BN1186 | [G/A] | C07 | 27694932 |
| BN1187 | [G/A] | C07 | 28040643 |
| BN1188 | [A/G] | C07 | 28767945 |
| BN1189 | [A/G] | C07 | 29073845 |
| BN1190 | [T/C] | C07 | 30029991 |
| BN1191 | [G/A] | C07 | 30705429 |
| BN1192 | [G/A] | C07 | 30947973 |
| BN1193 | [G/A] | C07 | 33513639 |
| BN1194 | [C/T] | C07 | 33621738 |
| BN1195 | [A/T] | C07 | 33988954 |
| BN1196 | [T/A] | C07 | 34326014 |
| BN1197 | [A/T] | C07 | 34684874 |
| BN1198 | [G/A] | C07 | 35023844 |
| BN1199 | [T/C] | C07 | 35229659 |
| BN1200 | [G/A] | C07 | 35670088 |
| BN1201 | [T/C] | C07 | 36617434 |
| BN1202 | [G/C] | C07 | 37313522 |
| BN1203 | [G/T] | C07 | 37535129 |
| BN1204 | [A/G] | C07 | 37759015 |
| BN1205 | [T/C] | C07 | 37935449 |
| BN1206 | [T/C] | C07 | 38473476 |
| BN1207 | [T/A] | C07 | 39355257 |
| BN1208 | [T/G] | C07 | 39744518 |
| BN1209 | [T/C] | C07 | 40863893 |
| BN1210 | [G/A] | C07 | 41326168 |
| BN1211 | [A/G] | C07 | 41673325 |
| BN1212 | [G/A] | C07 | 42388350 |
| BN1213 | [A/C] | C07 | 42591585 |
| BN1214 | [G/A] | C07 | 42907629 |
| BN1215 | [A/C] | C07 | 43183210 |
| BN1216 | [T/C] | C07 | 44040636 |
| BN1217 | [C/T] | C07 | 44224040 |
| BN1218 | [A/G] | C07 | 45160601 |
| BN1219 | [C/T] | C07 | 46953708 |
| BN1220 | [T/A] | C07 | 47928176 |
| BN1221 | [T/C] | C07 | 48555252 |
| BN1222 | [T/C] | C07 | 48754107 |
| BN1223 | [T/C] | C07 | 49153780 |
| BN1224 | [T/A] | C07 | 49608749 |
| BN1225 | [C/T] | C07 | 49827371 |
| BN1226 | [G/A] | C07 | 50030300 |
| BN1227 | [G/A] | C07 | 50580398 |
| BN1228 | [G/T] | C07 | 51101157 |
| BN1229 | [T/C] | C07 | 51215560 |
| BN1230 | [A/T] | C07 | 51820234 |
| BN1231 | [C/T] | C07 | 52183821 |
| BN1232 | [G/A] | C07 | 52832516 |
| BN1233 | [T/C] | C07 | 53819867 |
| BN1234 | [G/A] | C07 | 54220755 |
| BN1235 | [C/T] | C08 | 177880 |
| BN1236 | [T/A] | C08 | 388338 |
| BN1237 | [C/T] | C08 | 884352 |
| BN1238 | [A/T] | C08 | 1716856 |
| BN1239 | [G/A] | C08 | 2184665 |
| BN1240 | [T/C] | C08 | 2703255 |
| BN1241 | [G/A] | C08 | 2908013 |
| BN1242 | [A/G] | C08 | 3306204 |
| BN1243 | [T/C] | C08 | 3692903 |
| BN1244 | [A/G] | C08 | 4433900 |
| BN1245 | [T/C] | C08 | 5611113 |
| BN1246 | [T/C] | C08 | 6442533 |
| BN1247 | [G/A] | C08 | 6876605 |
| BN1248 | [T/A] | C08 | 7266091 |
| BN1249 | [T/C] | C08 | 8554852 |
| BN1250 | [C/T] | C08 | 8936830 |
| BN1251 | [T/A] | C08 | 11435202 |
| BN1252 | [T/A] | C08 | 14430095 |
| BN1253 | [C/T] | C08 | 14875646 |
| BN1254 | [A/C] | C08 | 15002413 |
| BN1255 | [T/C] | C08 | 15321671 |
| BN1256 | [G/C] | C08 | 15973314 |
| BN1257 | [G/C] | C08 | 17489863 |
| BN1258 | [G/A] | C08 | 18326046 |
| BN1259 | [G/A] | C08 | 18593854 |
| BN1260 | [C/T] | C08 | 18899610 |
| BN1261 | [G/A] | C08 | 19194404 |
| BN1262 | [G/A] | C08 | 19362263 |
| BN1263 | [G/A] | C08 | 19750124 |
| BN1264 | [A/C] | C08 | 20445933 |
| BN1265 | [C/G] | C08 | 21861820 |
| BN1266 | [G/T] | C08 | 22323771 |
| BN1267 | [G/T] | C08 | 24160742 |
| BN1268 | [C/T] | C08 | 24588948 |
| BN1269 | [A/T] | C08 | 25200160 |
| BN1270 | [C/T] | C08 | 25845594 |
| BN1271 | [A/G] | C08 | 25926150 |
| BN1272 | [C/T] | C08 | 26600972 |
| BN1273 | [T/C] | C08 | 27905734 |
| BN1274 | [C/A] | C08 | 28136954 |
| BN1275 | [T/A] | C08 | 28597508 |
| BN1276 | [G/A] | C08 | 29453028 |
| BN1277 | [G/A] | C08 | 30241749 |
| BN1278 | [A/G] | C08 | 30803076 |
| BN1279 | [G/A] | C08 | 30971031 |
| BN1280 | [A/G] | C08 | 31378887 |
| BN1281 | [G/A] | C08 | 31506095 |
| BN1282 | [T/G] | C08 | 31810252 |
| BN1283 | [G/A] | C08 | 32088223 |
| BN1284 | [A/G] | C08 | 32468571 |
| BN1285 | [C/T] | C08 | 32764069 |
| BN1286 | [G/A] | C08 | 33052855 |
| BN1287 | [A/C] | C08 | 33403297 |
| BN1288 | [T/A] | C08 | 35063901 |
| BN1289 | [C/G] | C08 | 36358337 |
| BN1290 | [G/T] | C08 | 36858692 |
| BN1291 | [A/C] | C08 | 37570513 |
| BN1292 | [C/T] | C08 | 37733876 |
| BN1293 | [C/T] | C08 | 38233207 |
| BN1294 | [C/T] | C08 | 38600698 |
| BN1295 | [C/G] | C08 | 39286345 |
| BN1296 | [C/A] | C08 | 39451976 |
| BN1297 | [C/T] | C08 | 39805862 |
| BN1298 | [G/A] | C08 | 40844780 |
| BN1299 | [T/C] | C09 | 1283762 |
| BN1300 | [C/T] | C09 | 2304641 |
| BN1301 | [A/G] | C09 | 2830555 |
| BN1302 | [A/C] | C09 | 3377206 |
| BN1303 | [G/A] | C09 | 3777259 |
| BN1304 | [A/G] | C09 | 4134282 |
| BN1305 | [T/C] | C09 | 4333788 |
| BN1306 | [G/A] | C09 | 4533109 |
| BN1307 | [C/T] | C09 | 4808041 |
| BN1308 | [G/A] | C09 | 5044005 |
| BN1309 | [C/A] | C09 | 5381471 |
| BN1310 | [C/T] | C09 | 6005186 |
| BN1311 | [G/A] | C09 | 6269584 |
| BN1312 | [C/T] | C09 | 7027094 |
| BN1313 | [C/T] | C09 | 7408170 |
| BN1314 | [C/T] | C09 | 7669880 |
| BN1315 | [A/G] | C09 | 8216737 |
| BN1316 | [A/G] | C09 | 8619683 |
| BN1317 | [T/C] | C09 | 9290670 |
| BN1318 | [G/A] | C09 | 9942436 |
| BN1319 | [C/T] | C09 | 10504604 |
| BN1320 | [C/T] | C09 | 10790093 |
| BN1321 | [G/T] | C09 | 11934178 |
| BN1322 | [A/T] | C09 | 12329017 |
| BN1323 | [G/T] | C09 | 12823226 |
| BN1324 | [G/A] | C09 | 13798833 |
| BN1325 | [C/T] | C09 | 14121467 |
| BN1326 | [T/C] | C09 | 14963317 |
| BN1327 | [A/G] | C09 | 15984783 |
| BN1328 | [C/T] | C09 | 17276337 |
| BN1329 | [G/A] | C09 | 17680228 |
| BN1330 | [C/A] | C09 | 18275408 |
| BN1331 | [G/A] | C09 | 18911713 |
| BN1332 | [G/C] | C09 | 19038837 |
| BN1333 | [T/C] | C09 | 19789379 |
| BN1334 | [A/G] | C09 | 20806485 |
| BN1335 | [T/A] | C09 | 21038568 |
| BN1336 | [A/C] | C09 | 21738410 |
| BN1337 | [A/G] | C09 | 22631400 |
| BN1338 | [G/A] | C09 | 22992740 |
| BN1339 | [G/A] | C09 | 24713578 |
| BN1340 | [T/A] | C09 | 25008931 |
| BN1341 | [A/T] | C09 | 25933263 |
| BN1342 | [C/T] | C09 | 26073109 |
| BN1343 | [G/A] | C09 | 26639966 |
| BN1344 | [G/A] | C09 | 26798967 |
| BN1345 | [G/T] | C09 | 28153609 |
| BN1346 | [G/A] | C09 | 28567797 |
| BN1347 | [T/A] | C09 | 29423970 |
| BN1348 | [A/G] | C09 | 29958682 |
| BN1349 | [C/T] | C09 | 30417856 |
| BN1350 | [T/C] | C09 | 31953801 |
| BN1351 | [C/T] | C09 | 32362479 |
| BN1352 | [T/G] | C09 | 33418713 |
| BN1353 | [G/C] | C09 | 33730552 |
| BN1354 | [C/T] | C09 | 36827877 |
| BN1355 | [A/C] | C09 | 38232699 |
| BN1356 | [T/A] | C09 | 39038814 |
| BN1357 | [T/C] | C09 | 40088426 |
| BN1358 | [A/C] | C09 | 40460087 |
| BN1359 | [T/G] | C09 | 41112813 |
| BN1360 | [C/T] | C09 | 41727885 |
| BN1361 | [G/A] | C09 | 42941490 |
| BN1362 | [G/C] | C09 | 43260317 |
| BN1363 | [A/G] | C09 | 44878151 |
| BN1364 | [A/G] | C09 | 45113275 |
| BN1365 | [G/A] | C09 | 46417761 |
| BN1366 | [T/C] | C09 | 46837315 |
| BN1367 | [C/T] | C09 | 47819202 |
| BN1368 | [T/C] | C09 | 48188055 |
| BN1369 | [A/G] | C09 | 48641036 |
| BN1370 | [G/A] | C09 | 48759717 |
| BN1371 | [G/C] | C09 | 49618393 |
| BN1372 | [A/G] | C09 | 50012536 |
| BN1373 | [G/A] | C09 | 50506492 |
| BN1374 | [C/T] | C09 | 50679051 |
| BN1375 | [C/A] | C09 | 51197804 |
| BN1376 | [A/G] | C09 | 52440542 |
| BN1377 | [T/G] | C09 | 52531485 |
| BN1378 | [G/A] | C09 | 52913562 |
| BN1379 | [T/G] | C09 | 53310301 |
| BN1380 | [T/C] | C09 | 53445902 |
| BN1381 | [A/G] | C09 | 54025969 |
| BN1382 | [T/A] | C09 | 55256069 |
| BN1383 | [G/A] | C09 | 56521841 |
| BN1384 | [A/G] | C09 | 57356786 |
| BN1385 | [T/C] | C09 | 57484709 |
| BN1386 | [A/G] | C09 | 57843296 |
| BN1387 | [A/G] | C09 | 58297949 |
| BN1388 | [C/A] | C09 | 58697511 |
| BN1389 | [A/G] | C09 | 59155815 |
| BN1390 | [G/A] | C09 | 59446910 |
| BN1391 | [A/T] | C09 | 60000799 |
| BN1392 | [A/T] | C09 | 60339769 |
| BN1393 | [C/T] | C09 | 60771136 |
| BN1394 | [T/A] | C09 | 61053345 |
| BN1395 | [G/A] | C09 | 61178632 |
| BN1396 | [A/G] | C09 | 61577411 |
| BN1397 | [C/T] | C09 | 62297148 |
| BN1398 | [C/T] | C09 | 62741728 |
| BN1399 | [C/G] | C09 | 63020374 |
| BN1400 | [G/T] | C09 | 63171947 |
| BN1401 | [A/C] | C09 | 63330083 |
| BN1402 | [A/G] | C09 | 63522057 |
| BN1403 | [T/A] | C09 | 63986029 |
| BN1404 | [A/G] | C09 | 64214135 |
| BN1405 | [T/C] | C09 | 64901846 |
| BN1406 | [A/T] | C09 | 65140654 |
| BN1407 | [G/A] | C09 | 66302905 |

2. An SNP chip of Brassica napus L., comprising at least one of a probe and a primer for detecting the SNP molecular marker combination of Brassica napus L. as claimed in claim 1.

3. A kit, comprising at least one of a probe and a primer for detecting the SNP molecular marker combination of Brassica napus L. as claimed in claim 1.

4. An application method of the SNP molecular marker combination of Brassica napus L. as claimed in claim 1, comprising:
genotyping the Brassica napus L. by using the SNP molecular marker combination.

5. An application method of the SNP molecular marker combination of Brassica napus L. as claimed in claim 1, comprising:
performing genome-wide association study on the Brassica napus L. by using the SNP molecular marker combination.

6. An application method of the SNP molecular marker combination of Brassica napus L. as claimed in claim 1, comprising:
performing cluster analysis on the Brassica napus L. by using the SNP molecular marker combination.

7. An application method of the SNP molecular marker combination of Brassica napus L. as claimed in claim 1, comprising:
performing paternity testing on the Brassica napus L. by using the SNP molecular marker combination.

8. An application method of the SNP molecular marker combination of Brassica napus L. as claimed in claim 1, comprising:
performing one of breeding and assisted breeding on Brassica napus L. by using the SNP molecular marker combination.

9. The application method as claimed in claim 8, wherein the one of breeding and assisted breeding comprises at least one selected from the group consisting of assisted major gene selection, backcrossing breeding and background selection, genomic selection breeding, molecular-assisted breeding, seed purity testing, genetically modified component identification, quantitative trait loci (QTL) analysis, species evolution analysis and germplasm resource identification.

10. A breeding method of Brassica napus L., comprising:
detecting deoxyribonucleic acid (DNA) of to-be-detected Brassica napus L. by using the SNP molecular marker combination of Brassica napus L. as claimed in claim 1, and selecting a target Brassica napus L. for subsequent breeding.
